(19) United States
(12) Patent Application Publication
(10) Pub. No.: US 2021/0214376 A1
(43) Pub. Date: Jul. 15, 2021

(54) HALOGENATED BIOTIN-MODIFIED DIMER AND USE THEREOF

(71) Applicants: The University of Tokyo, Tokyo (JP); SAVID THERAPEUTICS INC., Tokyo (JP)

(72) Inventors: Motomu KANAI, Tokyo (JP); Yohei SHIMIZU, Tokyo (JP); Kenzo YAMATSUGU, Tokyo (JP); Toshifumi TATSUMI, Tokyo (JP); Tatsuhiko KODAMA, Tokyo (JP); Akira SUGIYAMA, Tokyo (JP); Masanobu TSUKAGOSHI, Tokyo (JP)

(73) Assignees: THE UNIVERSITY OF TOKYO, Tokyo (JP); SAVID THERAPEUTICS INC., Tokyo (JP)

(21) Appl. No.: 17/059,334

(22) PCT Filed: May 30, 2019
(86) PCT No.: PCT/JP2019/021586

§ 371 Date: Nov. 27, 2020

(30) Foreign Application Priority Data
May 30, 2018 (JP) 2018-103808

(51) Int. Cl. C07D 519/00; A61K 49/14; A61K 49/08

(52) U.S. Cl. CPC C07D 519/00; A61K 49/085; A61K 49/14

(57) ABSTRACT

An object of the present invention is to provide a halogenated biotin-modified dimer, which is capable of imaging diagnosis or treatment by labeling with a halogen, a biotin-modified dimer having a high affinity for a mutant streptavidin with a low affinity for natural biotin. The present invention provides a compound represented by the following formula (1) or a salt thereof:

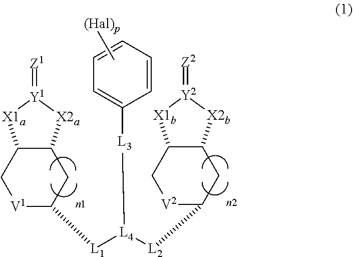

wherein the meaning of each symbol is the same as that described in the specification.

### TECHNICAL FIELD

The present invention relates to a halogenated biotin-modified dimer and the use thereof.

### BACKGROUND ART

Avidin and biotin, or streptavidin and biotin have an extremely high affinity between them (Kd=10⁻¹⁵ to 10⁻¹⁴ M). This is one of the strongest interactions between two biomolecules. At present, the interaction between avidin/streptavidin and biotin has been widely applied in the field of biochemistry, molecular biology, or medicine. A drug delivery method in which high binding ability between avidin/streptavidin and biotin is combined with an antibody molecule, namely, a pretargeting method has been devised.

Since chicken-derived avidin or microorganism-derived streptavidin exhibits high immunogenicity to a human body, such avidin or streptavidin is problematic in that an anti-avidin/streptavidin antibody is generated in an early stage after the administration thereof to a human body. This causes prevention of the practical use of the pretargeting method. Low immunogenic streptavidin that solves the aforementioned problem has been reported (International Publication WO2010/095455).

Low immunogenic streptavidin is characterized in that its immunogenicity to a human body is reduced. Since the low immunogenic streptavidin has an affinity for biotin existing in a human body, the low immunogenic streptavidin has been problematic in that it causes high background when used for diagnoses, or in that it is not likely to exhibit medicinal effects specifically on a disease when used for treatments.

Under such circumstances, a mutant streptavidin with a reduced affinity for natural biotin, and a modified biotin having a high affinity for the mutant streptavidin with a reduced affinity for natural biotin have been reported (International Publication WO2014/129446). Moreover, a mutant streptavidin with a reduced affinity for natural biotin, and a biotin-modified dimer having a high affinity for the mutant streptavidin with a low affinity for natural biotin have been reported (International Publication WO2015/125820).

### PRIOR ART DOCUMENTS

### Patent Documents

Patent Document 1: International Publication WO2010/095455

Patent Document 2: International Publication WO2014/129446

Patent Document 3: International Publication WO2015/125820

### SUMMARY OF INVENTION

### Object to be Solved by the Invention

It is an object of the present invention to provide a halogenated biotin-modified dimer, which is capable of imaging diagnosis or treatment by labeling with a halogen, a biotin-modified dimer having a high affinity for a mutant streptavidin with a low affinity for natural biotin. Furthermore, it is another object of the present invention to provide a diagnostic kit and/or a therapeutic kit, in which a combination of the above-described halogenated biotin-modified dimer and a mutant streptavidin is used.

### Means for Solving the Object

The present inventor has conducted intensive studies directed towards achieving the aforementioned objects. The inventor has succeeded in introducing a halogen into the biotin-modified dimer described in International Publication WO2015/125820. Then, the present inventor has confirmed that the obtained halogenated biotin-modified dimer binds, with a high affinity, to a mutant streptavidin having a low affinity for a natural biotin, thereby completing the present invention.

Specifically, the present invention provides the following inventions.

[1] A compound represented by the following formula (1) or a salt thereof:

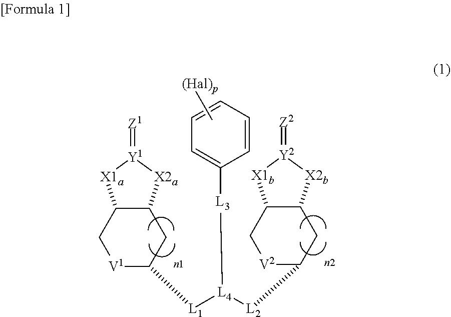

wherein
X1a, X1b, X2a and X2b each independently represent O or NH,
Y¹ and Y² each independently represent C or S,
Z¹ and Z² each independently represent O, S or NH,
V¹ and V² each independently represent S or S⁺—O⁻,
n1 and n2 each independently represent an integer of 0 or 1,
L₁, L₂, and L₃ each independently represent a divalent linking group,
L₄ represents a trivalent linking group,
Hal represents a halogen, and
p represents an integer of 1 to 5.
[2] The compound according to the above [1], wherein the compound represented by the formula (1) is a compound represented by the following formula (1a) or the following formula (1b):

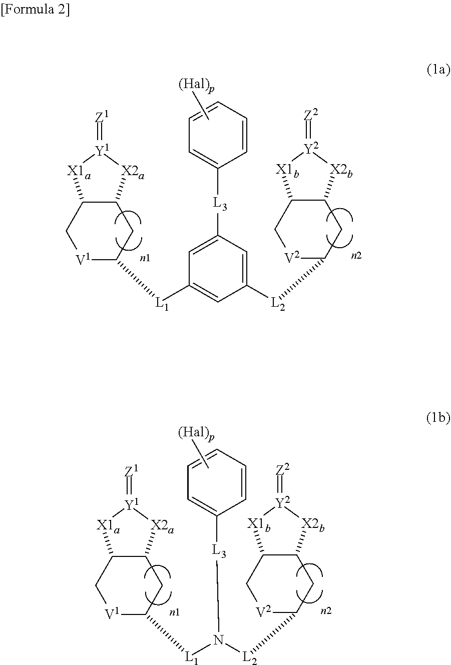

wherein each symbol is as defined in the above [1].
[3] The compound or a salt thereof according to the above [1] or [2], which is represented by the following formula (2) or the following formula (3), wherein n1 and n2 are 0.

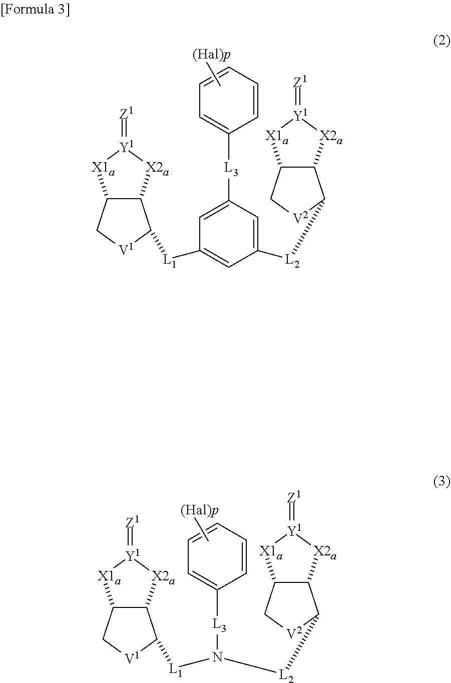

wherein each symbol is as defined in the above [1].
[4] The compound or a salt thereof according to any one of the above [1] to [3], wherein X1a, X1b, X2a and X2b represent NH, Y¹ and Y² represent C, Z¹ and Z² represent NH, and V¹ and V² represent S.
[5] The compound or a salt thereof according to any one of the above [1] to [4], wherein L₁, L₂, and L₃ each independently represent a divalent linking group consisting of a combination of groups selected from —CONH—, —NHCO—, —COO—, —OCO—, —CO—, —O—, and an alkylene group containing 1 to 10 carbon atoms.
[6] The compound or a salt thereof according to any one of the above [1] to [5], wherein
L₁ represents —(CH₂)ₘ₁-L₁₁-(CH₂)ₘ₂-L₁₂-*,
L₂ represents *-L₁₃-(CH₂)ₘ₃-L₁₄-(CH₂)ₘ₄—, and
L₃ represents L₁₅-L₂₁-L₁₆-,
wherein L₁₁, L₁₂, L₁₃, L₁₄, L₁₅, and L₁₆ each independently represent —CONH—, —NHCO—, —COO—, —OCO—, —CO—, or —O—,
m₁, m₂, m₃, and m₄ each independently represent an integer from 1 to 10,
* represents a binding site with a benzene ring,
L₂₁ represents —CH₂—(OCH₂)ₘ₅—, and
m₅ represents an integer from 1 to 10.
[7] The compound or a salt thereof according to any one of the above [1] to [6], wherein Hal represents I, ¹²⁵I, or ²¹¹At.
[8] The compound or a salt thereof according to any one of the above [1] to [7], wherein p is 1.
[9] A compound represented by any one of the following formulae:

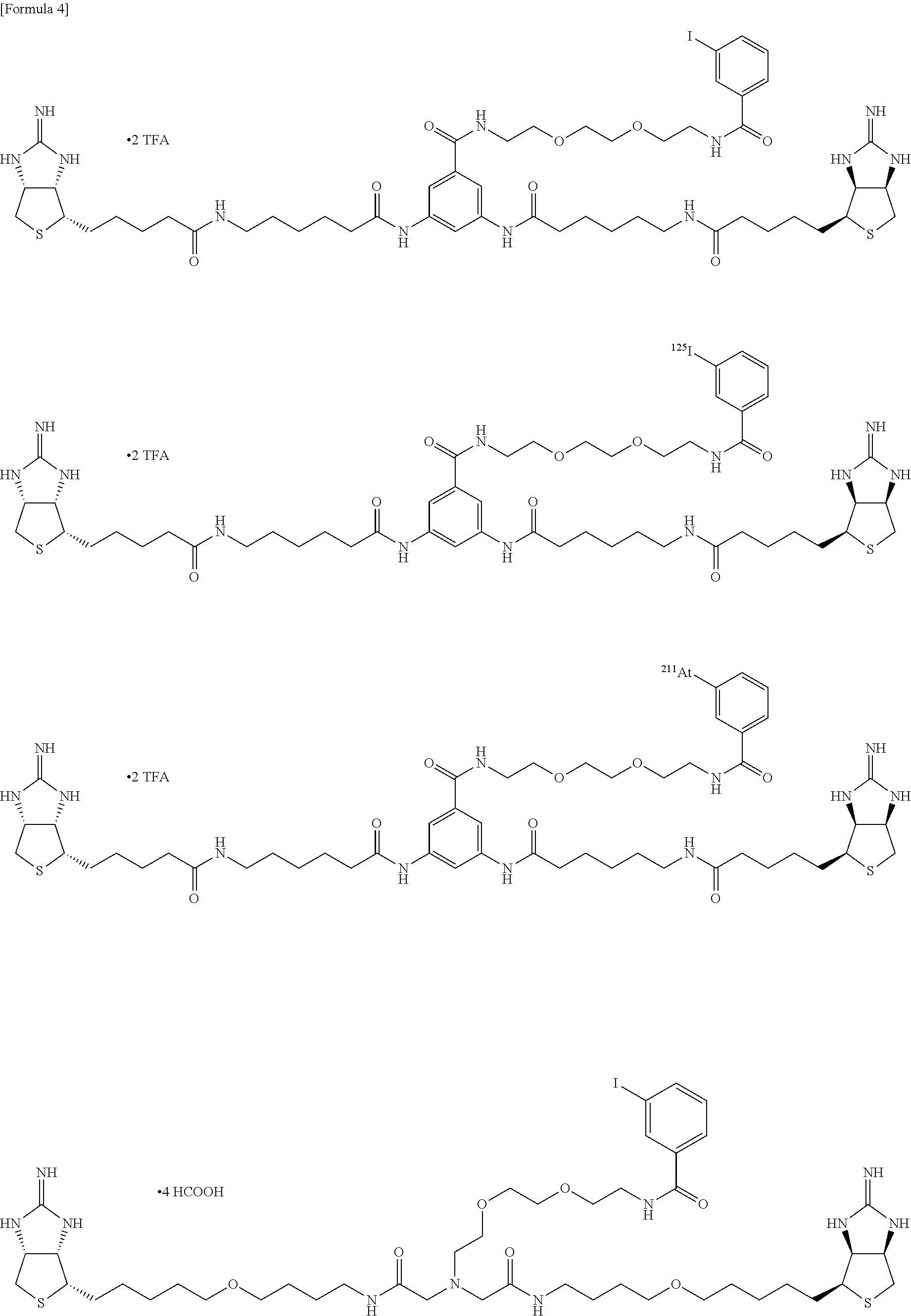

[10] A kit for treatment or diagnosis, comprising: (1) the compound according to any one of the above [1] to [9]; and (b) a mutant streptavidin-molecular probe conjugate obtained by binding a molecular probe to a mutant streptavidin comprising the amino acid sequence as set forth in SEQ ID NO: 19.
[11] A method for producing the compound or a salt thereof according to any one of the above [1] to [9], comprising reacting a compound represented by the following formula (A) with sodium halide or halogen:

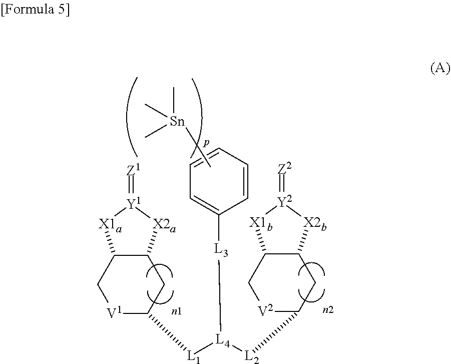

wherein each symbol is as defined in the above [1].
[12] A method for producing the compound or a salt thereof according to any one of the above [1] to [9], comprising reacting a compound represented by the following formula (B) with sodium halide or halogen:

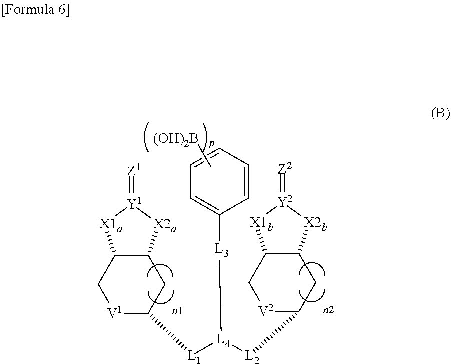

wherein each symbol is as defined in the above [1].

### Advantageous Effects of Invention

The halogenated biotin-modified dimer according to the present invention and a kit comprising the same are useful for diagnostic methods/therapeutic methods that are based on the pretargeting method.

### BRIEF DESCRIPTION OF DRAWINGS

FIG. 1 shows an outline of a domain structure.

FIG. 2 shows a CBB-stained SDS-PAGE electrophoretic pattern of CEA-V2122.

FIG. 3 shows a structural drawing of HER2-V2122.

FIG. 4 shows a CBB-stained SDS-PAGE electrophoretic pattern of HER2-V2122.

FIG. 5 shows the results obtained by confirming a binding conjugate of astatine-labeled Psyche B and Herceptin-Cupid, by using magnetic beads.

FIG. 6 shows the results obtained by confirming a binding conjugate of astatine-labeled Psyche B and Herceptin-Cupid, by using ovarian cancer cells SKOV3.

### EMBODIMENT OF CARRYING OUT THE INVENTION

Hereinafter, the present invention will be described in more detail.

### (1) Halogenated Biotin-Modified Dimer

The present invention is a compound represented by the following formula (1) or a salt thereof, and is preferably a compound represented by the following formula (1a), the following formula (1b), the following formula (2) or the following formula (3) or a salt thereof. The compound of the present invention is also referred to as a “halogenated biotin-modified dimer.”

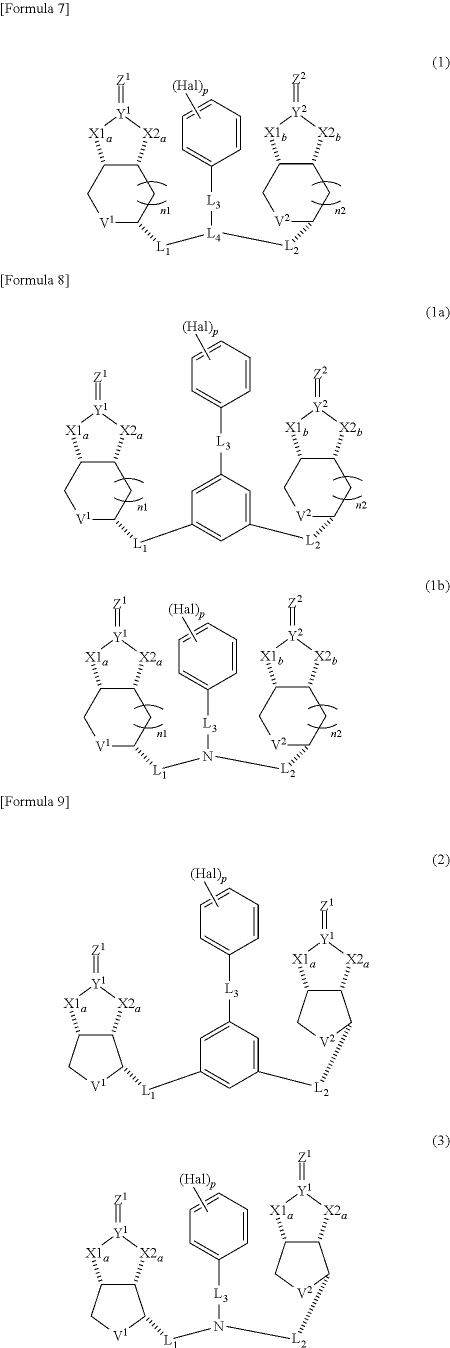

wherein
X1a, X1b, X2a and X2b each independently represent O or NH,
Y¹ and Y² each independently represent C or S,
Z¹ and Z² each independently represent O, S, or NH,
V¹ and V² each independently represent S or S⁺—O⁻,
n1 and n2 each independently represent an integer of 0 or 1,
L₁, L₂, and L₃ each independently represent a divalent linking group,
L₄ represents a trivalent linking group,
Hal represents a halogen, and
p represents an integer of 1 to 5.

In the formula (1), the formula (1a), the formula (1b), the formula (2), and the formula (3), the portions represented by the following structures:

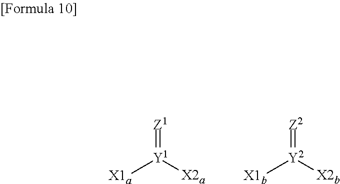

are preferably any one of the following portions, but are not limited thereto:

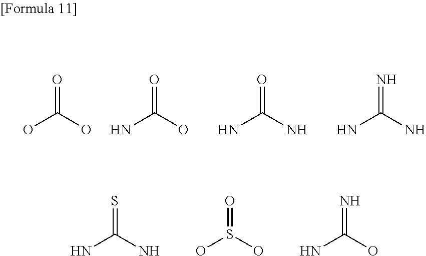

X1a, X1b, X2a and X2b preferably represent NH; Y¹ and Y² preferably represent C; Z¹ and Z² preferably represent NH; and V¹ and V² preferably represent S.

L₁, L₂, and L₃ each independently represent a divalent linking group consisting of a combination of groups selected from —CONH—, —NHCO—, —COO—, —OCO—, —CO—, —O—, and an alkylene group containing 1 to 10 carbon atoms.

L₁, L₂, and L₃ each independently represent a divalent linking group consisting of a combination of groups selected from —CONH—, —NHCO—, —O—, and an alkylene group containing 1 to 10 carbon atoms.

L₁ and L₂ each independently represent a divalent linking group consisting of a combination of groups selected from —CONH—, —NHCO—, and an alkylene group containing 1 to 10 carbon atoms.

L₁ preferably represents —(CH₂)ₘ₁-L₁-(CH₂)ₘ₂-L₁₂-*, L₂ preferably represents *-L₁₃-(CH₂)ₘ₃-L₁₄-(CH₂)ₘ₄—, and L₃ preferably represents L₁₅-L₂₁-L₁₆-.

L₁₁, L₁₂, L₁₃, L₁₄, L₁₅, and L₁₆ each independently represent —CONH—, —NHCO—, —COO—, —OCO—, —CO—, or —O—.

L₁₁ and L₁₂ are preferably —CONH—, —NHCO—, or —O—. Particularly preferably, L₁₁ is —O—, and L₁₂ is —NHCO—.

L₁₃ and L₁₄ are preferably —NHCO—, —CONH—, or —O—. Particularly preferably, L₁₃ is —CONH—, and L₁₄ is —O—.

L₁₅ is preferably —CONH— or a single bond.

L₁₆ is preferably —NHCO—.

m₁, m₂, m₃, and m₄ each independently represent an integer from 1 to 10, preferably an integer from 2 to 10, more preferably an integer from 2 to 8, and further preferably an integer from 2 to 6.

* represents a binding site with L₄,

L₂₁ preferably represents —(CH₂)₂—(O(CH₂)₂)ₘ₅—.

m₅ represents an integer from 1 to 10, preferably an integer from 1 to 6, and more preferably an integer from 1 to 4.

L₄ is a trivalent linking group, and is preferably

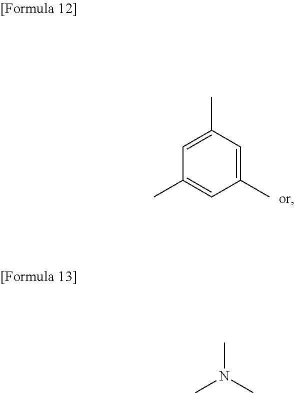

(which is a benzene-derived trivalent linking group or nitrogen atom).

Examples of the halogen represented by Hal may include fluorine (F), chlorine (Cl), bromine (Br), iodine (I), astatine (At), tennessine (Ts), and an isotope thereof. Preferred examples of the halogen may include iodine, astatine, and an isotope thereof. Specific examples of the halogen may include ¹²³I, ¹²⁴I, ¹²⁵I, ¹³¹I, ²¹⁰At, and ²¹¹At. Among the above-described halogens, I, ¹²³I, ¹²⁴I, ¹²⁵I, ¹³¹I, and ²¹¹At are preferable.

p represents an integer of 1 to 5, preferably an integer of 1 to 3, and particularly preferably 1.

### (2) Method for Producing Halogenated Biotin-Modified Dimer

As described in the Examples as described later, the halogenated biotin-modified dimer of the present invention can be produced by adding sodium halide (sodium iodide, Na¹²⁵I, etc.) or halogen (²¹¹At) dissolved in methanol to a solution of N-Bromo-succinimide or N-Iodo-succinimide in a mixture of methanol and acetic acid, then stirring the obtained mixture, and then adding a compound represented by the following formula (A) dissolved in methanol to the reaction mixture, so that the added compound is allowed to react with the reaction mixture. The additive amount of N-Bromo-succinimide or N-Iodo-succinimide is preferably 0.1 to 1.0 equivalent, more preferably 0.1 to 0.5 equivalents, and further preferably 0.1 to 0.3 equivalents. The additive amount (equivalent) of N-Bromo-succinimide or N-Iodo-succinimide is preferably set to be larger than the additive amount of sodium halide or halogen. However, if the additive amount of N-Bromo-succinimide or N-Iodo-succinimide is excessively large, the compound represented by the following formula (A) is likely to be decomposed. Thus, the additive amount of N-Bromo-succinimide or N-Iodo-succinimide is preferably adjusted to be an appropriate amount.

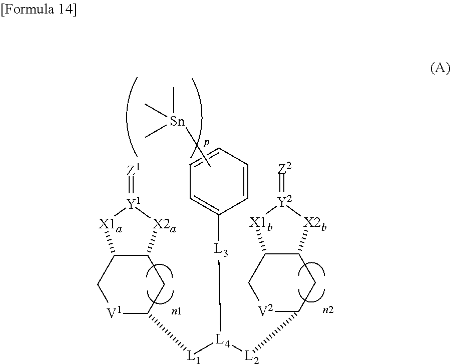

wherein each symbol is as defined in the formula (1).

Alternatively, as described in the Examples later, the halogenated biotin-modified dimer of the present invention can be produced by adding Tetrakis(pyridine)copper(II) triflate (whose additive amount is preferably 1 to 10 equivalents, and more preferably 2 to 8 equivalents) and 3,4,7,8-Tetramethyl-1,10-phenanthroline (whose additive amount is preferably 1 to 10 equivalents, and more preferably 2 to 8 equivalents) and sodium halide to a solution of a compound represented by the following formula (B) in a mixture of methanol and acetonitrile, so that they are allowed to react with one another.

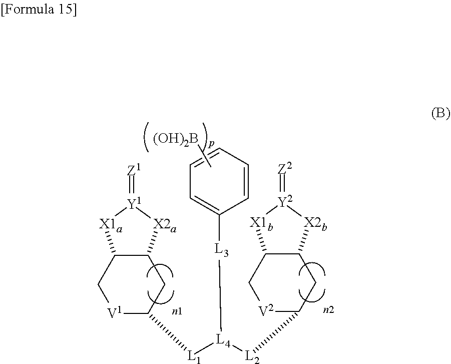

wherein each symbol is as defined in the formula (1).

### (3) Utilization of Halogenated Biotin-Modified Dimer

According to the present invention, provided is a therapeutic agent or a diagnostic agent, in which the halogenated biotin-modified dimer of the present invention is combined with a mutant streptavidin-molecular probe conjugate.

As mutant streptavidins used herein, the mutant streptavidins described in International Publication WO2014/129446 and International Publication WO2015/125820 can be used. Particularly preferably, a mutant streptavidin V2122 described in Example 3 of International Publication WO2015/125820 (SEQ ID NO: 4 of International Publication WO2015/125820) can be used.

Examples of the molecular probe used herein may include an antibody, a peptide, a nucleic acid, and an aptamer. Specifically, an antibody, a peptide, a nucleic acid, an aptamer, etc., which target the following antigens specifically expressed in cancer, can be used:

Epiregulin, ROBO 1, 2, 3, and 4, 1-40-β-amyloid, 4-1BB, 5AC, 5T4, ACVR2B, adenocarcinoma antigen, α-fetoprotein, angiopoetin 2, anthrax toxin, AOC3 (VAP-1), B-lymphoma cells, B7-H3, BAFF, β amyloid, C242 antigen, C5, CA-125, carbonic anhydrase 9 (CA-IX), cardiac myosin, CCL11 (eotaxin-1), CCR4, CCR5, CD11, CD18, CD125, CD140a, CD147 (basigin), CD147 (basigin), CD15, CD152, CD154 (CD40L), CD154, CD19, CD2, CD20, CD200, CD22, CD221, CD23 (IgE receptor), CD25 (IL-2 receptor α chain), CD28, CD3, CD30 (TNFRSF8), CD33, CD37, CD38 (cyclic ADP ribose hydrolase), CD4, CD40, CD41 (integrin α-IIb), CD44 v6, CD5, CD51, CD52, CD56, CD6, CD70, CD74, CD79B, CD80, CEA, CFD, ch4D5, CLDN18.2, Clostridium difficile, clumping factor A, CSF2, CTLA-4, cytomegalovirus, cytomegalovirus glycoprotein B, DLL4, DR5, E. coli Shiga toxin type 1, E. coli Shiga toxin type 2, EGFL7, EGFR, endotoxin, EpCAM, episialin, ERBB3, Escherichia coli, F protein of respiratory syncytial virus, FAP, fibrin II β chain, fibronectin extra domain-B, folate receptor 1, Frizzled receptor, GD2, GD3 ganglioside, GMCSF receptor α chain, GPNMB, hepatitis B surface antigen, hepatitis β virus, HER1, HER2/neu, HER3, HGF, HIV-1, HLA-DRβ, HNGF, Hsp90, human p amyloid, human scatter factor receptor kinase, human TNF, ICAM-1 (CD54), IFN-α, IFN-γ, IgE, IgE Fc region, IGF-1 receptor, IGF-I, IgG4, IGHE, IL-1β, IL-12, IL-13, IL-17, IL-17A, IL-22, IL-23, IL-4, IL-5, IL-6, IL-6 receptor, IL-9, ILGF2, influenza A hemagglutinin, insulin-like growth factor I receptor, integrin α4, integrin α4β7, integrin α5β1, integrin α7β7, integrin αIIbβ3, integrin αvβ3, integrin γ induced protein, interferon receptor, interferon α/β receptor, ITGA2, ITGB2 (CD18), KIR2D, L-selectin (CD62L), Lewis-Y antigen, LFA-1 (CD11a), lipoteichoic acid, LOXL2, LTA, MCP-1, mesothelin, MS4A1, MUC1, mucin CanAg, myostatin, N-glycolylneuraminic acid, NARP-1, NCA-90 (granulocyte antigen), NGF, NOGO-A, NRP1, Oryctolagus cuniculus, OX-40, oxLDL, PCSK9, PD-1, PDCD1, PDGF-R α, phosphatidylserine, prostate cancer cells, Pseudomonas aeruginosa, Rabies virus glycoprotein, RANKL, respiratory syncytial virus, RHD, Rh (Rhesus) factor, RON, RTN4, sclerostin, SDC1, selectin P, SLAMF7, SOST, sphingosine-1-phosphate, TAG-72, TEM1, tenascin C, TFPI, TGFβ1, TGFβ2, TGF-β, TNF-α, TRAIL-R1, TRAIL-R2, tumor antigen CTAA16.88, MUC1 tumor-specific glycosylation, TWEAK receptor, TYRP1 (glycoprotein 75), VEGF-A, VEGFR-1, VEGFR2, vimentin, and VWF.

A fusion body of a molecular probe such as a tumor antigen-specific antibody molecule and a mutant streptavidin is prepared, and the prepared fusion body is then administered to a patient, so that the mutant streptavidin can be accumulated specifically in cancer cells. Subsequently, the halogenated biotin-modified dimer of the present invention having an affinity for the above-described mutant streptavidin is administered to the patient, so that the halogen can be accumulated exactly in the cancer cells. In the present invention, the generation of an antibody is suppressed by a reduction in immunogenicity, and thereby, the clearance of the mutant streptavidin from the body in an early stage caused by the antibody, or shock such as anaphylaxis, can be prevented. Moreover, in the present invention, using the mutant streptavidin of the present invention as an in-vitro diagnostic agent or a clinical diagnostic agent, regarding which the tissue, serum and the like collected from patients are used, noise derived from biotin or a biotin-binding protein present in the tissue, serum and the like can be reduced, so that diagnosis and examination with a higher S/N ratio can be carried out.

Otherwise, in the present invention, a conjugate is prepared by binding a fusion body of a molecular probe such as a tumor antigen-specific antibody molecule and a mutant streptavidin with the halogenated biotin-modified dimer of the present invention, and the thus prepared conjugate can be administered to a patient.

Various types of molecules can be used as antibodies that are to be bound to the mutant streptavidin. Either a polyclonal antibody or a monoclonal antibody may be used. The subclass of the antibody is not particularly limited. Preferably, IgG, and particularly preferably, IgG₁ is used. Furthermore, the term “antibody” includes all of modified antibodies and antibody fragments. Examples of such an antibody include, but are not limited to: a humanized antibody; a human type antibody; a human antibody; antibodies from various types of animals such as a mouse, a rabbit, a rat, a guinea pig and a monkey; a chimeric antibody between a human antibody and an antibody from a different type of animal; diabody; scFv; Fd; Fab; Fab′; and F(ab)′₂.

The conjugate of the mutant streptavidin and the antibody can be obtained by applying a method known to persons skilled in the art. For example, such a conjugate can be obtained by a chemical bond method (U.S. Pat. No. 5,608,060). Alternatively, DNA encoding the mutant streptavidin is ligated to DNA encoding an antibody, and using an expression vector or the like, the ligated DNA is then expressed in a host cell, so that such a conjugate can be obtained in the form of a fusion protein. The DNA encoding the mutant streptavidin may be ligated to the DNA encoding an antibody via DNA encoding a suitable peptide, called a linker. The mutant streptavidin-molecular probe conjugate is desirably produced, while keeping the specific binding force between an antibody and a target molecule.

The present invention will be more specifically described in the following examples. However, these examples are not intended to limit the scope of the present invention.

### EXAMPLES

### Synthesis of Halogenated Biotin-Modified Dimer Compound

### General Methods

Nuclear magnetic resonance (NMR) spectra were measured using a JEOL ECX500 (¹H NMR: 500 MHz) or JEOL ECS400 (H NMR: 400 MHz) spectrometer. Chemical shift was expressed in ppm as a value with respect to the residual solvent peak in a deuterated solvent as an internal reference (CDCl₃: δ=7.26 ppm, CD₃OD: δ=3.31 ppm). Low-resolution mass spectra (LHMS) were measured using ESI-MS according to Shimadzu LCMS-2020 System. The reaction was traced by thin-layer chromatography (TLC) or low-resolution mass spectrometry (LRMS).

Reverse phase high performance liquid chromatography (HPLC) was carried out using JASCO-HPLC System. Detection was conducted using ultraviolet ray at a wavelength of 254 nm, and a gradient solvent system (acetonitrile/0.1% trifluoroacetic acid MQ solution or 0.1% formic acid MQ solution) was used as a mobile phase. The analysis was carried out using a YMC-Triart-C18 (150 mm×4.6 mm I.D.) column at a flow rate of 1 mL/min. Fractionation was carried out using a YMC-Triart-C18 (250 mm×10 mm I.D. or 150 mm×4.6 mm I.D.) column at a flow rate of 6.3 mL/min in the case of the former column or at a flow rate of 1 mL/min in the case of the latter column.

### Example 1

### 1

### (3aS,3a′S,4S,4′S,6aR,6a′R)-4,4′-(((((5-((2-(2-(2-(3-(Trimethylstannyl)benzamido)ethoxy)ethoxy)ethyl)carbamoyl)-1,3-phenylene)bis(azanediyl))bis(6-oxohexane-6,1-diyl))bis(azanediyl))bis(5-oxopentane-5,1-diyl))bis(tetrahydro-1H-thieno[3,4-d]imidazol-2(3H)-iminium) formate

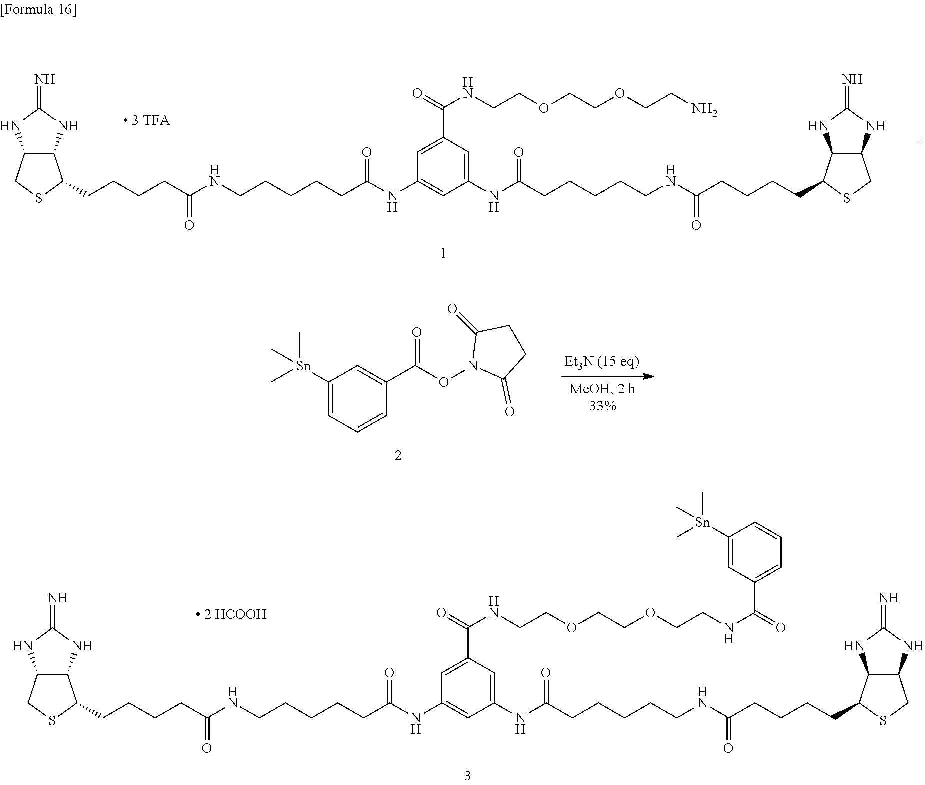 [SMILES: N=C1N[C@H]2[C@H](CS[C@H]2CCCCC(=O)NCCCCCC(=O)Nc2cc(NC(=O)CCCCCNC(=O)CCCC[C@@H]3SC[C@@H]4NC(=N)N[C@@H]43)cc(C(=O)NCCOCCOCCN)c2)N1 ; [CH3][Sn]([CH3])([CH3])[c]1cccc(C(=O)ON2C(=O)CCC2=O)c1 ; [CH3][Sn]([CH3])([CH3])[c]1cccc(C(=O)NCCOCCOCCNC(=O)c2cc(NC(=O)CCCCCNC(=O)CCCC[C@@H]3SC[C@@H]4NC(=N)N[C@@H]43)cc(NC(=O)CCCCCNC(=O)CCCC[C@@H]3SC[C@@H]4NC(=N)N[C@@H]43)c2)c1]

Compound 2 (19.3 mg, 51 μmol) and triethylamine (47.3 μl, 510 μmol) were added to a solution of bisiminobiotin 1 (International Publication WO2015/125820; JP Patent Publication (Kokai) No. 2017-66155 A) (44.7 mg, 34 μmol) in methanol (1 ml), and the obtained mixture was then stirred at room temperature for 2 hours. Thereafter, the solvent was removed under reduced pressure, and the obtained crude product was purified by reverse phase HPLC (gradient: 2% for 2 min; 2-100% for 90 min CH₃CN in 0.1% HCOOH aqueous solution, retention time=43.6 min, YMC-Triart C18, flow rate=6.3 ml/min) to obtain the title compound 3 (15.0 mg, a yield of 33%, a white amorphous substance).

¹H NMR (500 MHz, CD₃OD) δ: 0.29 (s, 9H), 1.35-1.45 (m, 8H), 1.48-1.80 (m, 16H), 2.18 (t, 4H, J=7.4 Hz), 2.37 (t, 4H, J=7.4 Hz), 2.80 (d, 2H, J=13.2 Hz), 2.97 (dd, 2H, J=5.2 Hz, 13.2 Hz), 3.24-3.29 (m, 2H), 3.50-3.58 (m, 4H), 3.62-3.67 (m, 8H), 4.50 (dd, 2H, J=4.6 Hz, 8.0 Hz), 4.70 (dd, 2H, J=4.6 Hz, 8.0 Hz), 7.37 (t, 1H, J=7.4 Hz), 7.62 (d, 1H, J=6.9 Hz), 7.70 (d, 2H, J=2.1 Hz), 7.92-7.94 (m, 1H), 8.01 (t, 1H, J=1.7 Hz), 8.51-8.57 (m, 1H). LRMS (ESI): m/z 614 [M+2H]²⁺.

### 2

### (3aS,3a′S,4S,4′S,6aR,6a′R)-4,4′-(((((5-((2-(2-(2-(3-Iodobenzamido)ethoxy)ethoxy)ethyl)carbamoyl)-1,3-phenylene)bis(azanediyl))bis(6-oxohexane-6,1-diyl))bis(azanediyl))bis(5-oxopentane-5,1-diyl))bis(tetrahydro-1H-thieno[3,4-d]imidazol-2(3H)-iminium) 2,2,2-trifluoroacetate

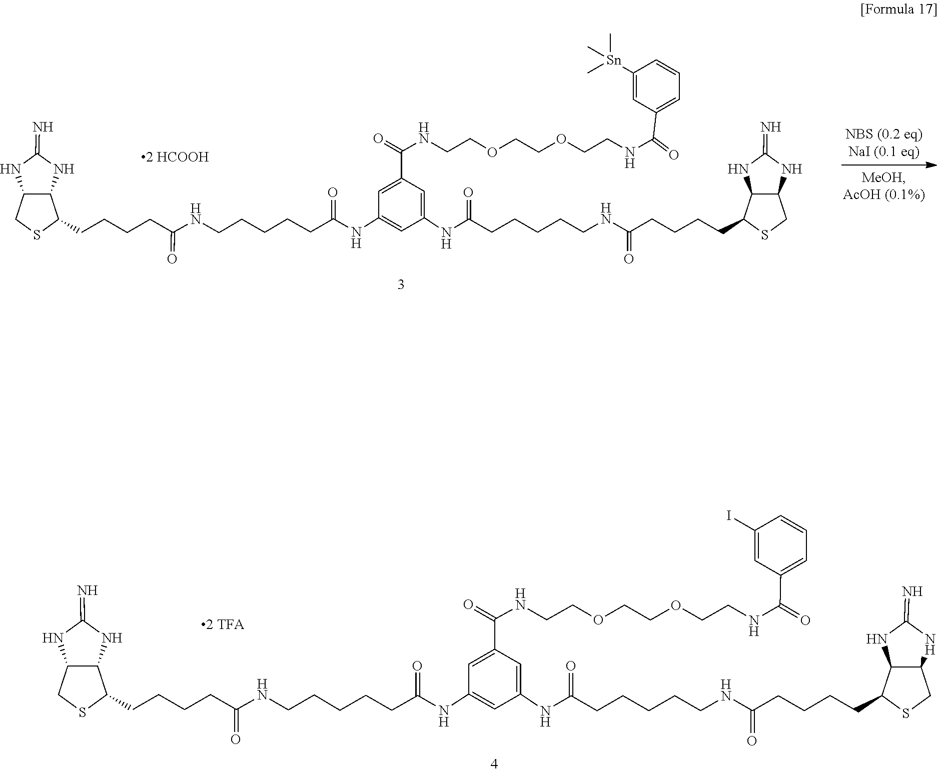 [SMILES: [CH3][Sn]([CH3])([CH3])[c]1cccc(C(=O)NCCOCCOCCNC(=O)c2cc(NC(=O)CCCCCNC(=O)CCCC[C@@H]3SC[C@@H]4NC(=N)N[C@@H]43)cc(NC(=O)CCCCCNC(=O)CCCC[C@@H]3SC[C@@H]4NC(=N)N[C@@H]43)c2)c1 ; N=C1N[C@H]2[C@H](CS[C@H]2CCCCC(=O)NCCCCCC(=O)Nc2cc(NC(=O)CCCCCNC(=O)CCCC[C@@H]3SC[C@@H]4NC(=N)N[C@@H]43)cc(C(=O)NCCOCCOCCNC(=O)c3cccc(I)c3)c2)N1]

Sodium iodide (0.00114 mg, 0.076 μmol) dissolved in methanol (100 μl) was added to a solution of N-Bromo-succinimide (0.0027 mg, 0.152 μmol) in a mixture of methanol (560 μl) and acetic acid (0.76 μl), and the obtained mixture was then stirred at room temperature for 5 minutes. Thereafter, bisiminobiotin 3 (0.1 mg, 0.076 μmol) dissolved in methanol (100 μl) was added to the reaction solution, and the obtained mixture was then stirred at room temperature for 10 minutes. The reaction solution was not concentrated, but was purified by reverse phase HPLC (gradient: 30% for 2 min; 30-100% for 20 min CH₃CN in 0.1% CF₃COOH aqueous solution, retention time=8.5 min, YMC-Triart C18, flow rate=1.0 ml/min) to obtain the title compound 4 (a yellow highly viscous oily substance). The obtained product was identical to a preparation synthesized by an alternative method, in terms of the retention time and the measured mass, according to LC-MS analysis.

The title compound 4 is also referred to as “Psyche B-iodine.”

¹H NMR (400 MHz, CDCl₃) δ: 1.34-1.75 (m, 24H), 2.18 (t, 4H, J=7.2 Hz), 2.38 (t, 4H, J=7.2 Hz), 2.80 (d, 2H, J=13.0 Hz), 2.98 (dd, 2H, J=4.9 Hz, 13.5 Hz), 3.18 (t, 4H, J=7.2 Hz), 3.23-3.28 (m, 2H), 3.55 (t, 4H, J=5.4 Hz), 3.63-3.70 (m, 8H), 4.50 (dd, 2H, J=4.5 Hz, 8.1 Hz), 4.70 (dd, 2H, J=4.5 Hz, 8.1 Hz), 7.19 (t, 1H, J=7.6 Hz), 7.70 (d, 2H, J=1.8 Hz), 7.76-7.80 (m, 1H), 7.83-7.87 (m, 1H), 7.98 (t, 1H, J=1.8 Hz), 8.15 (t, 1H, 1.8 Hz). LRMS (ESI): m/z 595 [M+2H]²⁺.

### 3

### (3aS,3a′S,4S,4′S,6aR,6a′R)-4,4′-(((((5-((2-(2-(2-(3-(Iodo-¹²⁵I)benzamido)ethoxy)ethoxy)ethyl)carbamoyl)-1,3-phenylene)bis(azanediyl))bis(6-oxohexane-6,1-diyl))bis(azanediyl))bis(5-oxopentane-5,1-diyl))bis(tetrahydro-1H-thieno[3,4-d]imidazol-2(3H)-iminium) 2,2,2-trifluoroacetate

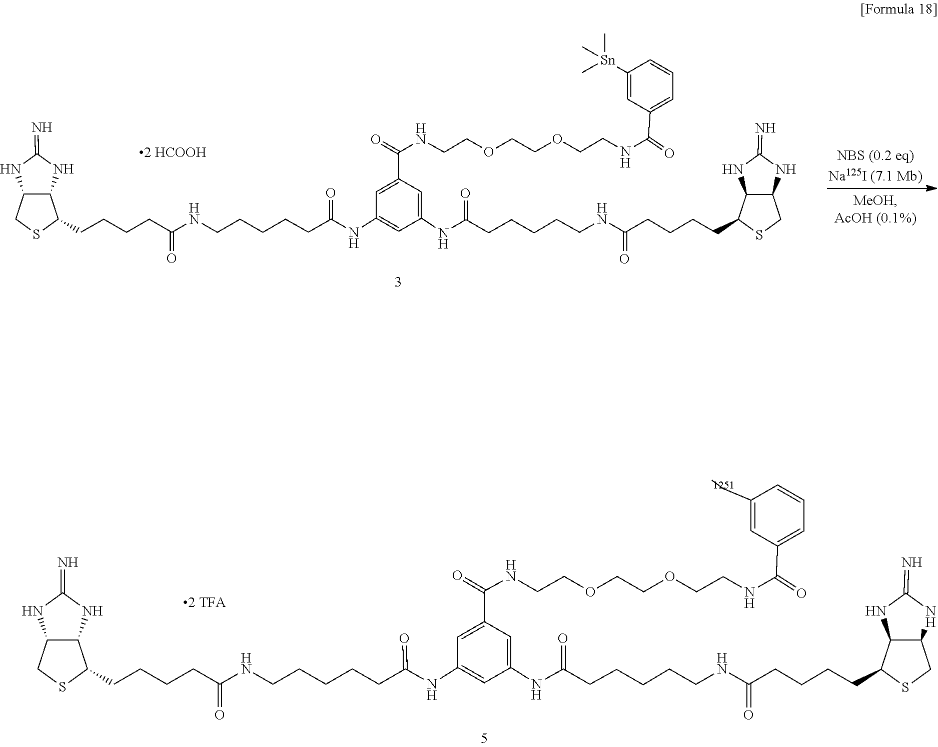

Na¹²⁵I (7.1 MBq) dissolved in methanol (100 μl) was added to a solution of N-Bromosuccinimide (0.00271 mg, 0.152 μmol) in a mixture of methanol (560 μl) and acetic acid (0.76 μl), and the obtained mixture was then stirred at room temperature for 5 minutes. Thereafter, bisiminobiotin 3 (0.1 mg, 0.076 μmol) dissolved in methanol (100 μl) was added to the reaction solution, and the obtained mixture was then stirred at room temperature for 10 minutes. Thereafter, the reaction solution was not concentrated, but was purified by reverse phase HPLC (30% for 2 min; 30-100% for 20 min CH₃CN in 0.1% CF₃COOH aqueous solution, retention time=8.5 min, YMC-Triart C18, flow rate=1.0 m₁/min) to obtain the title compound 5.

### 4

### (3aS,3a′S,4S,4′S,6aR,6a′R)-4,4′-(((((5-((2-(2-(2-(3-(Astato-²¹¹At)benzamido)ethoxy)ethoxy)ethyl)carbamoyl)-1,3-phenylene)bis(azanediyl))bis(6-oxohexane-6,1-diyl))bis(azanediyl))bis(5-oxopentane-5,1-diyl))bis(tetrahydro-1H-thieno[3,4-d]imidazol-2(3H)-iminium) 2,2,2-trifluoroacetate

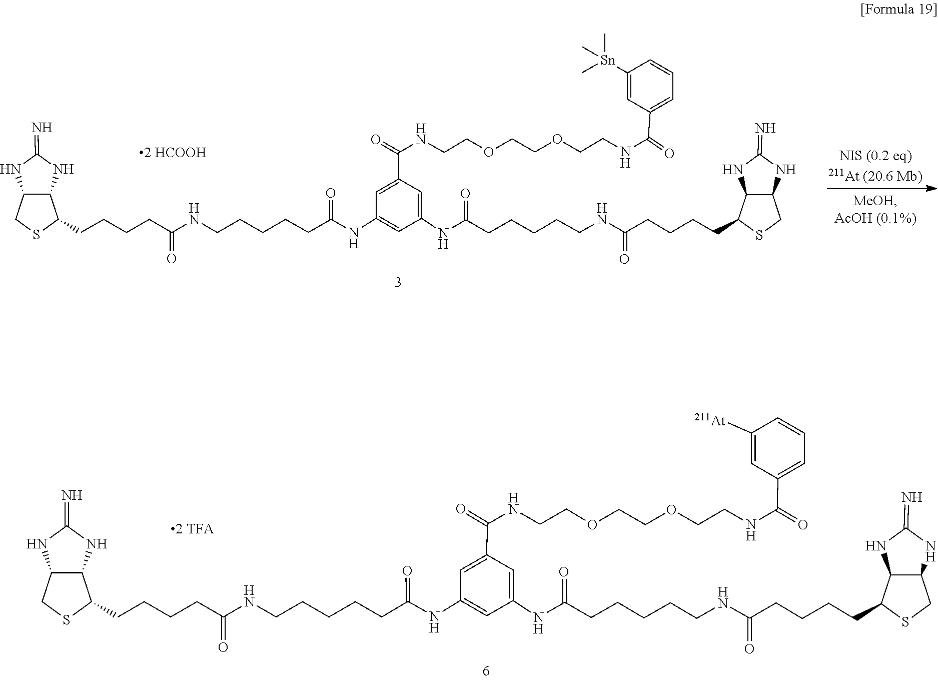

²¹¹At (20.6 MBq) dissolved in methanol (100 μl) was added to a solution of N-Iodosuccinimide (0.0034 mg, 0.152 μmol) in a mixture of methanol (560 μl) and acetic acid (0.76 μl), and the obtained mixture was then stirred at room temperature for 1 minute. Thereafter, bisiminobiotin 3 (0.1 mg, 0.076 μmol) dissolved in methanol (100 μl) was added to the reaction solution, and the obtained mixture was then stirred at room temperature for 10 minutes. Thereafter, the reaction solution was not concentrated, but was purified by reserve phase HPLC (30% for 2 min; 30-80% for 13 min CH₃CN in 0.1% CF₃COOH aqueous solution, retention time=7.0 min, YMC-Triart C18, flow rate=1.0 m₁/min) to obtain the title compound 6. The title compound 6 is also referred to as “Psyche B-At211.”

### Example 2

### 1

### 2,5-Dioxopyrrolidin-1-yl 3-(4,4,5,5-tetramethyl-1,3,2-dioxaborolan-2-yl)benzoate

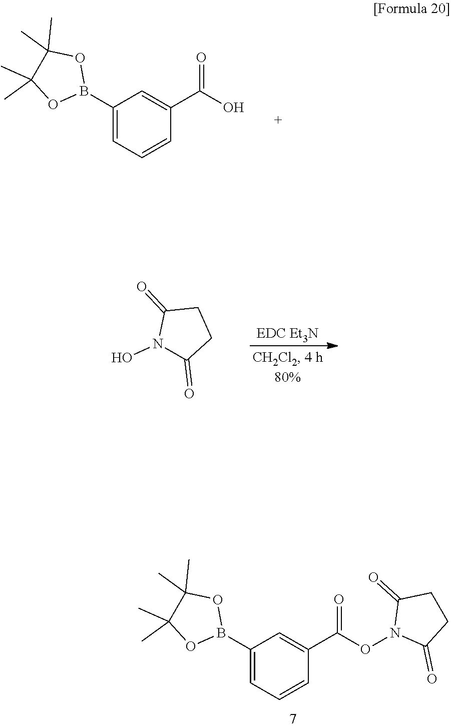 [SMILES: CC1(C)OB(c2cccc(C(=O)O)c2)OC1(C)C ; O=C1CCC(=O)N1O ; CC1(C)OB(c2cccc(C(=O)ON3C(=O)CCC3=O)c2)OC1(C)C]

N-Hydroxysuccinimide (21.4 mg, 188 μmol) and 1-(3-Dimethylaminopropyl)-3-ethylcarbodiimide Hydrochloride (36 mg, 188 mol) were added to a solution of 3-(4,4,5,5-Tetramethyl-1,3,2-dioxaborolan-2-yl)benzoic acid in a mixture of dichloromethane (3 ml) and triethylamine (26 μl, 188 μmol), and the obtained mixture was then stirred in an argon atmosphere at room temperature for 4 hours. Thereafter, the solvent was removed under reduced pressure, and water was added to the residue, followed by extraction with ethyl acetate. The organic layer was washed with a saturated ammonium chloride aqueous solution once, then with a saturated sodium hydrogen carbonate aqueous solution once, and then with a saturated saline once. The resultant was dried over sodium sulfate, and the solvent was then removed under reduced pressure to obtain the target compound 7 (42.7 mg, a yield of 80%, a white solid).

¹H NMR (400 MHz, CDCl₃) δ: 1.35 (s, 12H), 2.85-2.98 (br, 4H), 7.52 (t, 1H, J=7.6 Hz), 8.06-8.10 (m, 1H), 8.20 (dt, 1H, J=1.8 Hz, J=7.6 Hz), 8.59 (s, 1H). LRMS (ESI): m/z 368 [M+Na]⁺.

### 2

### (3-((2-(2-(2-(3,5-bis(6-(5-((3aS,4S,6aR)-2-Iminohexahydro-1H-thieno[3,4-d]imidazol-4-yl)pentanamido)hexanamido)benzamido)ethoxy)ethoxy)ethyl)carbamoyl)phenyl)boronic acid

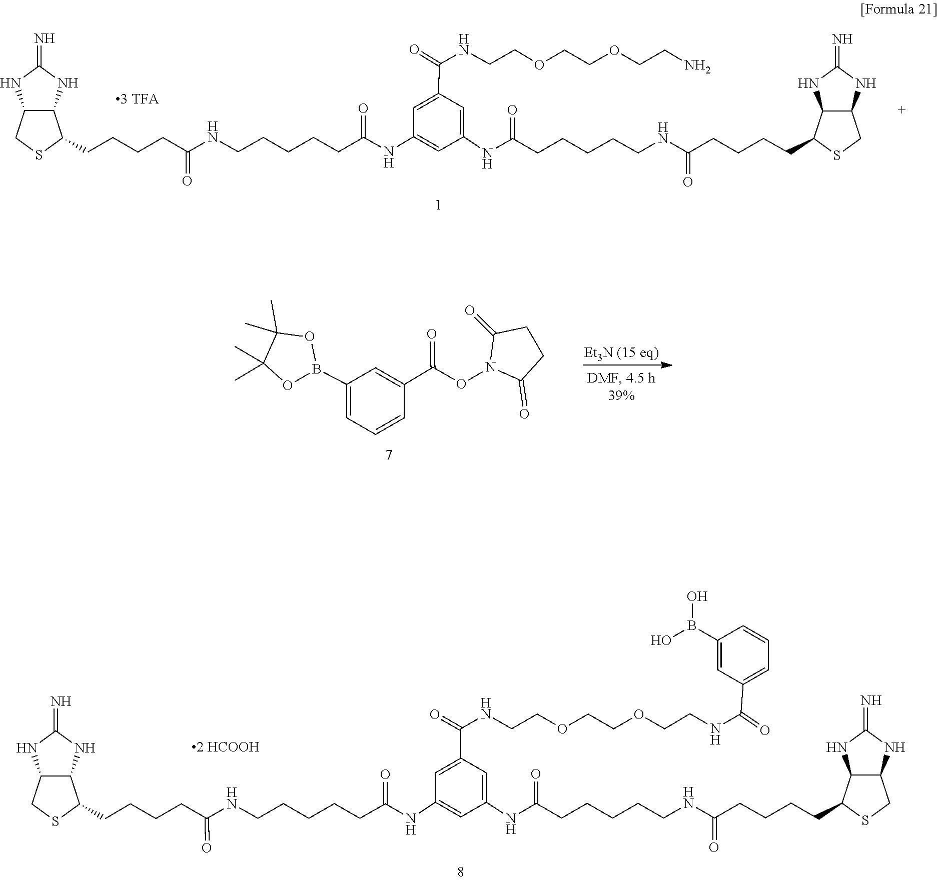 [SMILES: N=C1N[C@H]2[C@H](CS[C@H]2CCCCC(=O)NCCCCCC(=O)Nc2cc(NC(=O)CCCCCNC(=O)CCCC[C@@H]3SC[C@@H]4NC(=N)N[C@@H]43)cc(C(=O)NCCOCCOCCN)c2)N1 ; CC1(C)OB(c2cccc(C(=O)ON3C(=O)CCC3=O)c2)OC1(C)C ; N=C1N[C@H]2[C@H](CS[C@H]2CCCCC(=O)NCCCCCC(=O)Nc2cc(NC(=O)CCCCCNC(=O)CCCC[C@@H]3SC[C@@H]4NC(=N)N[C@@H]43)cc(C(=O)NCCOCCOCCNC(=O)c3cccc(B(O)O)c3)c2)N1]

Compound 7 (3.9 mg, 11.5 μmol) and triethylamine (16 μl, 115 μmol) were added to a solution of bisiminobiotin 1 (International Publication WO2015/125820; JP Patent Publication (Kokai) No. 2017-66155 A) (10 mg, 7.68 μmol) in DMF (400 μl), and the obtained mixture was then stirred at room temperature for 4.5 hours. Thereafter, the solvent was removed under reduced pressure, and the obtained crude product was then purified by reverse phase HPLC (gradient: 0% for 5 min; 0-100% for 90 min CH₃CN in 0.1% HCOOH aqueous solution, retention time=32.5 min, YMC-Triart C18, flow rate=3.5 ml/min) to obtain the title compound 8 (3.6 mg, a yield of 39%, a white amorphous substance).

¹H NMR (500 MHz, CDCl₃) δ: 1.34-1.75 (m, 24H), 2.17 (t, 4H, J=7.4 Hz), 2.37 (t, 4H, J=7.4 Hz), 2.79 (d, 2H, J=13.2 Hz), 2.96 (dd, 2H, J=4.6 Hz, 13.2 Hz), 3.17 (t, 4H, J=6.3 Hz), 3.21-3.28 (m, 2H), 3.50-3.57 (m, 4H), 3.61-3.69 (m, 8H), 4.49 (dd, 2H, J=4.0 Hz, 7.4 Hz), 4.69 (dd, 2H, J=4.6 Hz, 8.0 Hz), 7.35 (t, 1H, J=7.4 Hz), 7.65-7.79 (m, 3H), 7.96-8.03 (m, 2H), 8.50 (s, 1H). LRMS (ESI): m/z 554 [M+2H]²⁺.

### 3

### (3aS,3a′S,4S,4′S,6aR,6a′R)-4,4′-(((((5-((2-(2-(2-(3-Iodobenzamido)ethoxy)ethoxy)ethyl)carbamoyl)-1,3-phenylene)bis(azanediyl))bis(6-oxohexane-6,1-diyl))bis(azanediyl))bis(5-oxopentane-5,1-diyl))bis(tetrahydro-1H-thieno[3,4-d]imidazol-2(3H)-iminium) 2,2,2-trifluoroacetate

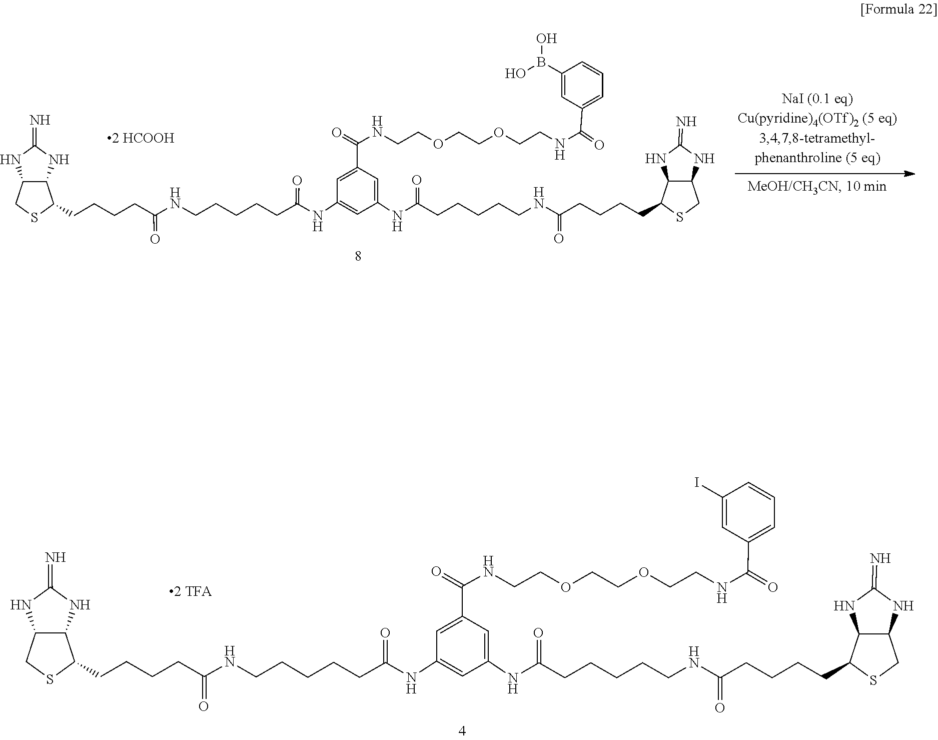 [SMILES: N=C1N[C@H]2[C@H](CS[C@H]2CCCCC(=O)NCCCCCC(=O)Nc2cc(NC(=O)CCCCCNC(=O)CCCC[C@@H]3SC[C@@H]4NC(=N)N[C@@H]43)cc(C(=O)NCCOCCOCCNC(=O)c3cccc(B(O)O)c3)c2)N1 ; N=C1N[C@H]2[C@H](CS[C@H]2CCCCC(=O)NCCCCCC(=O)Nc2cc(NC(=O)CCCCCNC(=O)CCCC[C@@H]3SC[C@@H]4NC(=N)N[C@@H]43)cc(C(=O)NCCOCCOCCNC(=O)c3cccc(I)c3)c2)N1]

Tetrakis(pyridine)copper(II) triflate (2.8 mg, 4.2 μmol), 3,4,7,8-Tetramethyl-1,10-phenanthroline (0.99 mg, 4.2 μmol) and sodium iodide (0.0125 mg, 0.0834 μmol) were added to a solution of bisiminobiotin 8 in a mixture of methanol (17.5 μl) and acetonitrile (2.5 μl), and the obtained mixture was then stirred at room temperature for 10 minutes. Thereafter, the reaction solution was subjected to LCMS analysis, and as a result, the reaction solution was identical to a preparation synthesized by an alternative method, in terms of retention time and measured mass.

The title compound 4 is also referred to as “Psyche B-iodine.”

### Example 3

### Methyl 5-((2S,3S,4R)-3,4-diaminotetrahydrothiophen-2-yl)pentanoate hydrobromide

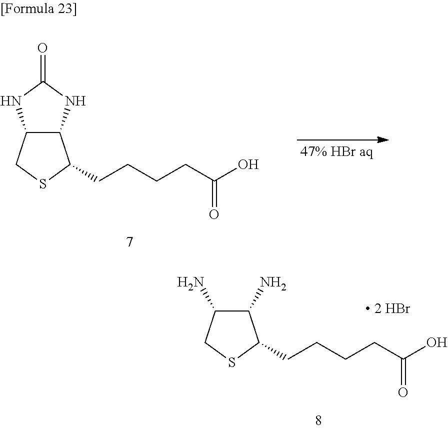 [SMILES: O=C(O)CCCC[C@@H]1SC[C@@H]2NC(=O)N[C@@H]21 ; N[C@@H]1[C@H](CCCCC(=O)O)SC[C@@H]1N]

12 ml of a 47% Hydrogen bromide aqueous solution was added to biotin 7 (2.5 g, 10.2 mmol), and the obtained mixture was then stirred under reflux in an argon atmosphere at 150° C. for 48 hours. Thereafter, the solvent was removed under reduced pressure. The obtained crude product 8 was used in the subsequent reaction, without being subjected to further purification operations.

### Methyl 5-((2S,3S,4R)-3,4-diaminotetrahydrothiophen-2-yl)pentanoate hydrobromide

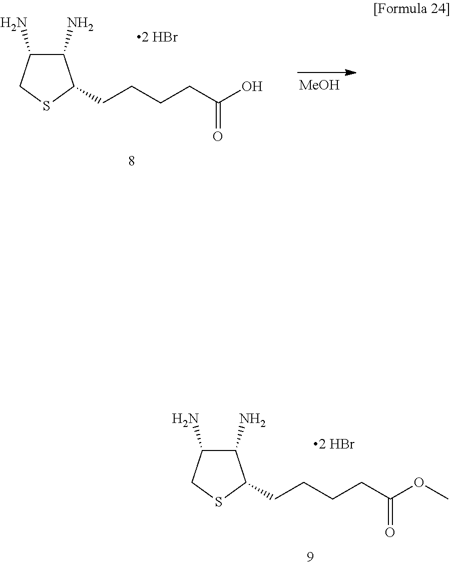

20 ml of Methanol was added to the crude product 8, and the obtained mixture was then stirred under reflux in an argon atmosphere for 5 hours. Thereafter, the solvent was removed under reduced pressure, and the obtained crude product 9 was used in the subsequent reaction, without being subjected to further purification operations.

### 5-((2S,3S,4R)-3,4-Diaminotetrahydrothiophen-2-yl)pentan-1-ol

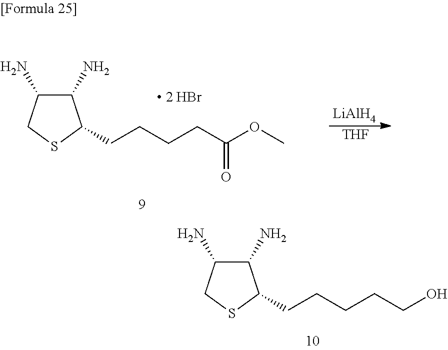

Under cooling on ice, the crude product 9 in a THF (400 ml) solution was added dropwise to a solution of lithium aluminum hydride (1.55 g, 40.9 mmol) in THF (50 ml) over 30 minutes. The obtained mixture was stirred in an argon atmosphere on an ice bath for 3 hours. Thereafter, water (1.5 ml), a 15% NaOH aqueous solution (1.5 ml), and water (4.5 ml) were added to the reaction mixture, and a filtrate obtained by sodium sulfate filtration was then concentrated under reduced pressure. The obtained roughly purified product 10 (1.9 g, a yellow solid) was used in the subsequent reaction, without being subjected to further purification operations.

### Di-tert-butyl ((2S,3S,4R)-2-(5-hydroxypentyl)tetrahydrothiophene-3,4-diyl)dicarbamate

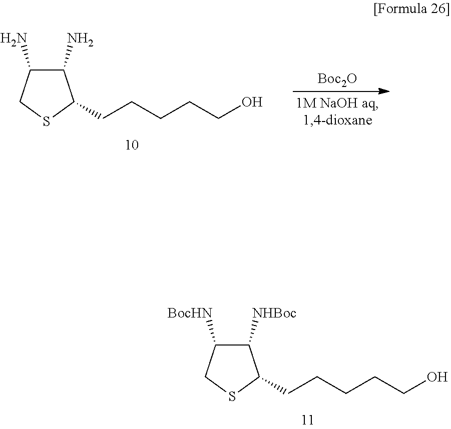 [SMILES: "Boc2O" ; N[C@@H]1[C@H](CCCCCO)SC[C@@H]1N ; CC(C)(C)OC(=O)N[C@H]1[C@@H](NC(=O)OC(C)(C)C)CS[C@H]1CCCCCO]

Di-tert-butyl dicarbonate (4.26 g, 20.5 mmol) was added to a solution of the crude product 10 in 1,4-dioxane (20 ml) at room temperature, and then, a 1 M NaOH aqueous solution (20 ml) was added thereto under cooling on ice. The obtained mixture was stirred in an argon atmosphere at room temperature for 15 hours. Thereafter, the solvent was removed under reduced pressure, and a 1 M HCl aqueous solution was then added to the residue, followed by extraction with ethyl acetate. The organic layer was washed with a saturated saline. The resultant was dried over sodium sulfate, and the solvent was then removed under reduced pressure. The obtained roughly purified product was purified by silica gel chromatography (hexane/ethyl acetate=67:33→20:80→0:100→ethyl acetate/methanol=95:5→90:10) to obtain the target compound 11 (1.77 g, a yield of 46% after 4 stages from the compound 7, a white amorphous substance).

¹H NMR (500 MHz, CDCl₃) δ: 1.20-1.38 (m, 4H), 1.38-1.50 (m, 19H), 1.50-1.57 (m, 2H), 1.57-1.78 (m, 1H), 2.45 (t, J=10.3 Hz, 1H), 3.10-3.27 (m, 1H), 3.45-3.46 (m, 1H), 3.60 (t, J=6.9 Hz, 2H), 4.05-4.25 (m, 1H), 4.25-4.40 (m, 1H), 4.70-4.85 (m, 1H), 4.90-5.05 (m, 1H). LRMS (ESI): m/z 427 [M+H]⁺.

### Di-tert-butyl ((2S,3S,4R)-2-(5-oxopentyl)tetrahydrothiophene-3,4-diyl)dicarbamate

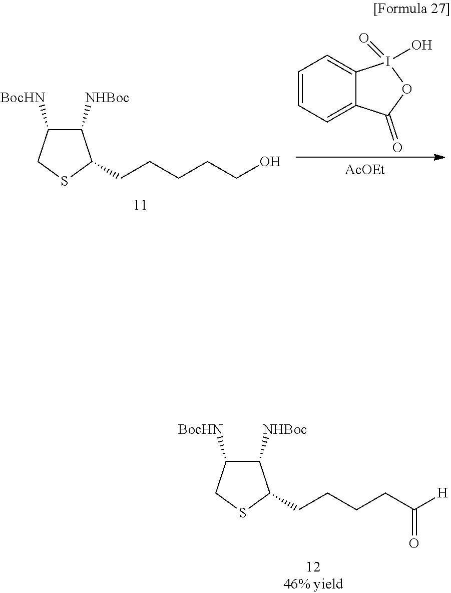 [SMILES: "AcOEt" ; CC(C)(C)OC(=O)N[C@H]1[C@@H](NC(=O)OC(C)(C)C)CS[C@H]1CCCCCO ; O=C1OI(=O)(O)c2ccccc21 ; CC(C)(C)OC(=O)N[C@H]1[C@@H](NC(=O)OC(C)(C)C)CS[C@H]1CCCCC=O]

2-Iodoxybenzoic acid (4.08 g, 5.68 mmol, purity: 39%, a commercially available product) was added to a solution of the compound 11 (1.15 g, 2.84 mmol) in ethyl acetate (12 ml). The obtained mixture was stirred in an argon atmosphere at 80° C. under reflux for 4 hours. Thereafter, the solution was filtrated with Celite, and the filtrate was then removed under reduced pressure. The obtained crude product was purified by silica gel chromatography (hexane/ethyl acetate=76:24→55:45) to obtain the target compound 12 (0.53 g, a yield of 46%, a white amorphous substance).

¹²H NMR (500 MHz, CD₃OD) δ: 1.30-1.50 (m, 24H), 2.42 (t, J=5.7 Hz, 2H), 2.47 (t, J=10.3 Hz, 1H), 3.15-3.25 (m, 1H), 3.45-3.55 (m, 1H), 4.10-4.35 (m, 2H), 4.65-4.80 (m, 1H), 4.90-5.00 (m, 1H), 9.75 (t, J=1.7 Hz, 1H).

### Di-tert-butyl ((2S,3S,4R)-2-(5,5-bis(4-(1,3-dioxoisoindolin-2-yl)butoxy)pentyl)tetrahydrothiophene-3,4-diyl)dicarbamate

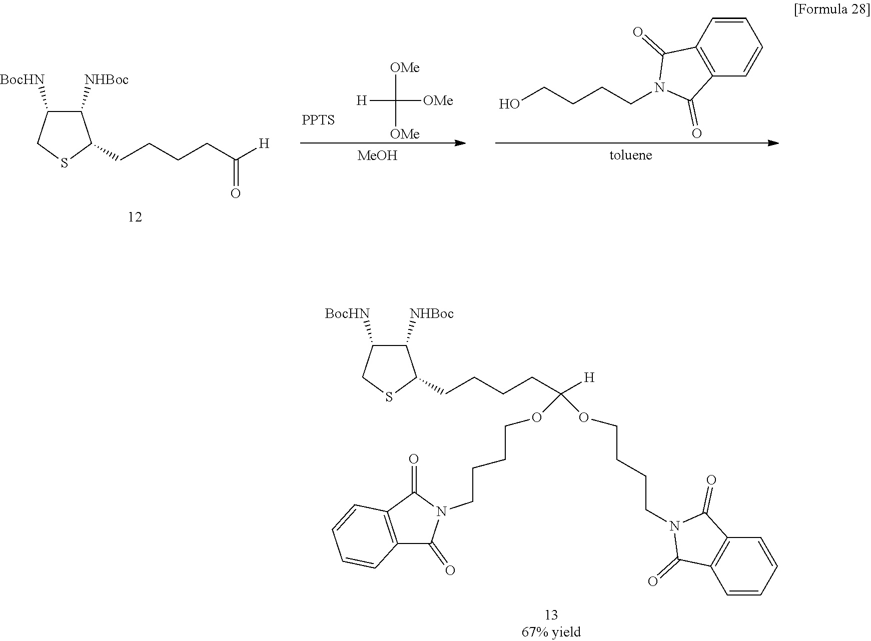 [SMILES: "MeOH" ; CC(C)(C)OC(=O)N[C@H]1[C@@H](NC(=O)OC(C)(C)C)CS[C@H]1CCCCC=O ; "PPTS" ; COC(OC)OC ; O=C1c2ccccc2C(=O)N1CCCCO ; CC(C)(C)OC(=O)N[C@H]1[C@@H](NC(=O)OC(C)(C)C)CS[C@H]1CCCCC(OCCCCN1C(=O)c2ccccc2C1=O)OCCCCN1C(=O)c2ccccc2C1=O]

Methyl orthoformate (333 μl, 2.95 mmol) and pyridinium p-toluenesulfonate (13.6 mg, 0.059 mmol) were added to a solution of the compound 12 (235.7 mg, 0.59 mmol) in methanol (2 ml) at room temperature. The obtained mixture was stirred in an argon atmosphere at room temperature for 10 hours, and the solvent and the methyl orthoformate were removed under reduced pressure. Toluene (13 ml) and 2-(4-Hydroxybutyl)isoindoline-1,3-dione (1.03 g. 4.68 mmol) were added to the residue, and the thus obtained mixture was then stirred using a Dean-stark device under reflux for 8 hours. Thereafter, the solvent was removed under reduced pressure, and the obtained crude product was purified by silica gel chromatography (hexane/ethyl acetate=64:36→46:54→35:65) to obtain the target compound 13 (0.32 g, a yield of 67%).

¹H NMR (500 MHz, CDCl₃) δ: 1.28-1.80 (m, 34H), 2.46 (t, J=10.3 Hz, 1H), 3.05-3.35 (m, 1H), 3.34-3.44 (m, 2H), 3.47-3.62 (m, 3H), 3.70, (t, J=7.4 Hz, 4H), 4.18-4.23 (m, 1H), 4.28-4.33 (m, 1H), 4.39 (t, J=5.2 Hz, 1H), 4.74-4.81 (m, 1H), 4.97-5.03 (m, 1H), 7.68-7.74 (m, 4H), 7.80-7.84 (m, 4H).

### 2-(4-((5-((2S,3S,4R)-3,4-Diaminotetrahydrothiophen-2-yl)pentyl)oxy)butyl)isoindoline-1,3-dione

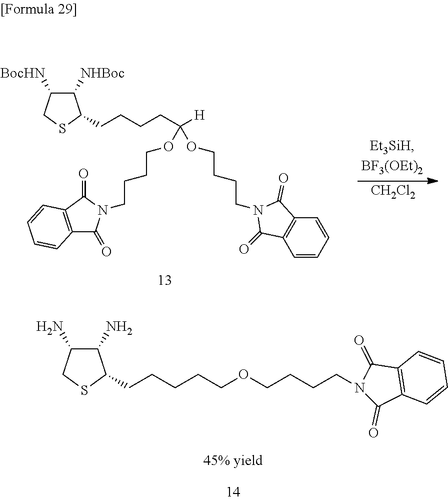 [SMILES: CC(C)(C)OC(=O)N[C@H]1[C@@H](NC(=O)OC(C)(C)C)CS[C@H]1CCCCC(OCCCCN1C(=O)c2ccccc2C1=O)OCCCCN1C(=O)c2ccccc2C1=O ; "CH2Cl2" ; N[C@@H]1[C@H](CCCCCOCCCCN2C(=O)c3ccccc3C2=O)SC[C@@H]1N]

Triethylsilane (468 μl, 2.93 mmol) and boron trifluoride diethyl etherate (234 μl, 0.881 mmol) were added to the compound 13 (302 mg, 0.367 mmol) in a dichloromethane (7.2 ml) solution at room temperature, and the obtained mixture was then stirred in an argon atmosphere at room temperature for 11 hours. Thereafter, methanol (500 μl) was added to the reaction solution, and the obtained mixture was then stirred at room temperature for 1 hour. After that, the solvent was removed under reduced pressure. The obtained crude product was purified by silica gel chromatography (chloroform/methanol=99:1→75:25→65:35). A saturated sodium bicarbonate solution was added to the obtained compound, and the obtained mixture was then extracted with dichloromethane. The extract was dried over sodium sulfate, and the solvent was then removed under reduced pressure to obtain the target compound 14 (67.6 mg, a yield of 45%, a yellow oily substance).

¹H NMR (400 MHz, CDCl₃) δ: 1.32-1.62 (m, 9H), 1.67-1.76 (m, 3H), 2.55 (d, J=13.2 Hz, 1H), 2.93 (dd, J=5.7 Hz, 12.6 Hz, 1H), 3.20-3.30 (m, 1H), 3.41 (t, J=6.3 Hz, 2H), 3.45 (t, J=6.3 Hz, 2H), 3.68 (t, J=6.9 Hz, 2H) 3.82 (t, J=5.7 Hz, 1H), 3.94 (t, J=6.3 Hz, 1H), 7.76-7.81 (m, 2H), 7.82-7.87 (m, 2H). LRMS (ESI): m/z 406 [M+H]⁺.

### tert-Butyl ((3aS,4S,6aR,E)-4-(5-(4-(1,3-dioxoisoindolin-2-yl)butoxy)pentyl)tetrahydro-1H-thieno[3,4-d]imidazol-2(3H)-ylidene)carbamate

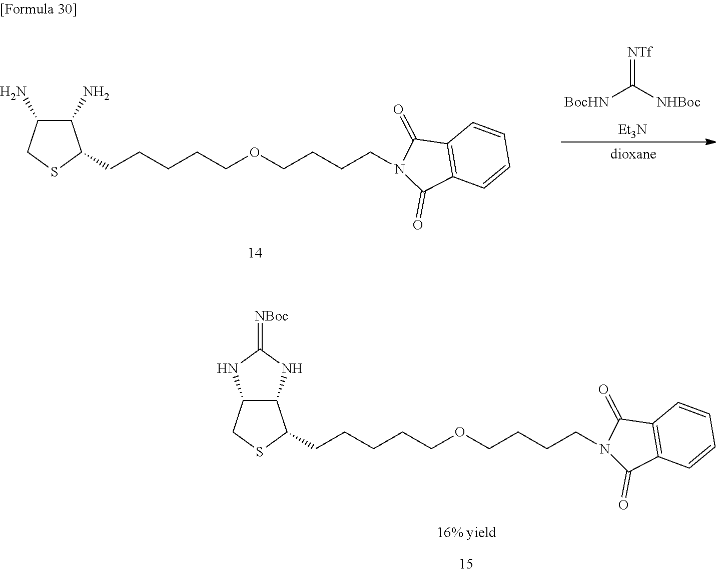

A Goodman reagent (42 mg, 0.107 mmol) dissolved in dioxane (400 μl) was added to a solution of the compound 14 (43.5 mg, 0.107 mmol) in a mixture of dioxane (300 μl) and triethylamine (30 μl, 0.214 mmol) at room temperature. The obtained mixture was stirred in an argon atmosphere at room temperature for 21 hours, and the solvent was then removed under reduced pressure. The obtained crude product was purified by silica gel chromatography (chloroform/methanol=100:0→10:1→5:1) to obtain the target compound 15 (9.1 mg, a yield of 16%).

¹H NMR (500 MHz, CDCl₃) δ: 1.32-1.50 (m, 4H), 1.43 (s, 9H), 1.51-1.69 (m, 5H), 1.71-1.81 (m, 3H), 2.86-2.95 (m, 2H), 3.18-3.24 (m, 1H), 3.38 (t, J=6.3 Hz, 2H), 3.42 (t, J=6.3 Hz, 2H), 3.71 (t, J=6.9 Hz, 2H), 4.42 (dd, J=5.2 Hz, 8.0 Hz, 1H),4.62 (m, 1H), 7.67-7.73 (m, 2H), 7.80-7.86 (m, 2H). LRMS (ESI): m/z 531 [M+H]⁺.

### tert-Butyl ((3aS,4S,6aR,E)-4-(5-(4-aminobutoxy)pentyl)tetrahydro-1H-thieno[3,4-d]imidazol-2(3H)-ylidene)carbamate

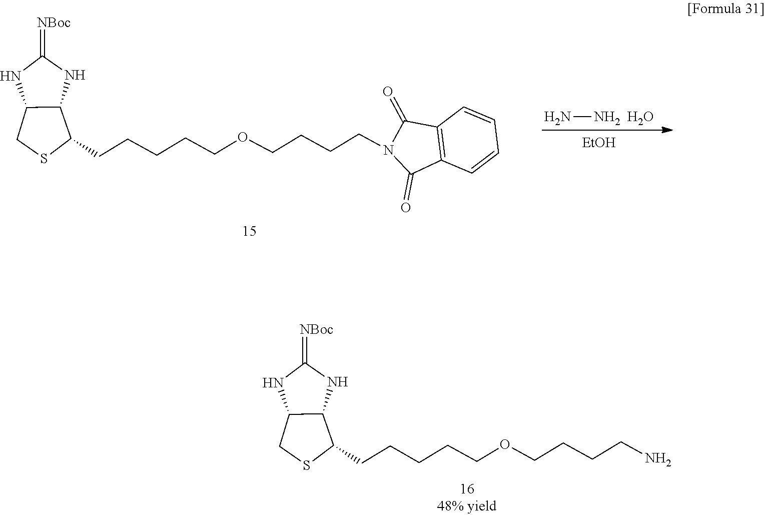

Hydrazine hydrate (10 mg, 0.3 mmol) was added to a solution of the compound 15 (31.7 mg, 0.06 mmol) in ethanol (500 μl) at room temperature. The reaction solution was stirred at 50° C. for 10 hours. Thereafter, the reaction solution was subjected to suction filtration, and a filtrate was then removed under reduced pressure. The obtained crude product was purified by silica gel chromatography (amino silica, chloroform/methanol=100:0→94:6) to obtain the target compound 16 (11.8 mg, a yield of 49%).

¹H NMR (400 MHz, CDCl₃) δ: 1.40-1.50 (m, 13H), 1.51-1.69 (m, 7H), 1.72-1.82 (m, 1H), 2.69 (t, J=6.7 Hz, 2H), 2.81 (d, J=6.3 Hz, 1H), 2.97 (dd, J=4.9 Hz, 13.0 Hz, 1H), 3.24-3.32 (m, 1H), 3.40-3.48 (m, 4H), 4.41 (dd, J=4.5 Hz, 8.1 Hz, 1H), 4.62 (dd, J=4.5 Hz, 8.1 Hz, 1H). LRMS (ESI): m/z 401 [M+H]⁺.

### 14-(Carboxymethyl)-2,2-dimethyl-4-oxo-3,8,11-trioxa-5,14-diazahexadecan-16-oic Acid

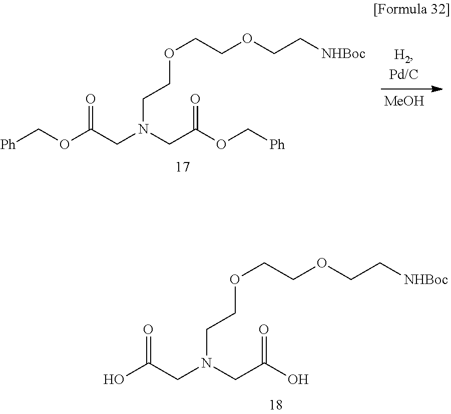 [SMILES: "MeOH" ; CC(C)(C)OC(=O)NCCOCCOCCN(CC(=O)OCc1ccccc1)CC(=O)OCc1ccccc1 ; CC(C)(C)OC(=O)NCCOCCOCCN(CC(=O)O)CC(=O)O]

10% Palladium carbon (24.1 mg, 22.6 μmol) was added to a solution of the compound 17 (AMGEN INC. WO2006/14645, 2006, A1) (131.1 mg, 240.7 mmol) in methanol (5 ml), and the obtained mixture was then stirred in a hydrogen atmosphere at room temperature for 3 hours. Thereafter, the reaction solution was filtrated with Celite, and the solvent was then removed under reduced pressure. The obtained crude product 18 (93.3 mg, yield: quant., a colorless highly-viscous oily substance) was used in the subsequent reaction, without being subjected to further purification operations.

### tert-Butyl (22-((3aS,4S,6aR,E)-2-((Tert-butoxycarbonyl)imino)hexahydro-1H-thieno[3,4-d]imidazol-4-yl)-9-(2-((4-((5-((3aS,4S,6aR,Z)-2-((tert-butoxycarbonyl)imino)hexahydro-1H-thieno[3,4-d]imidazol-4-yl)pentyl)oxy)butyl)amino)-2-oxoethyl)-11-oxo-3,6,17-trioxa-9,12-diazadocosyl)carbamate

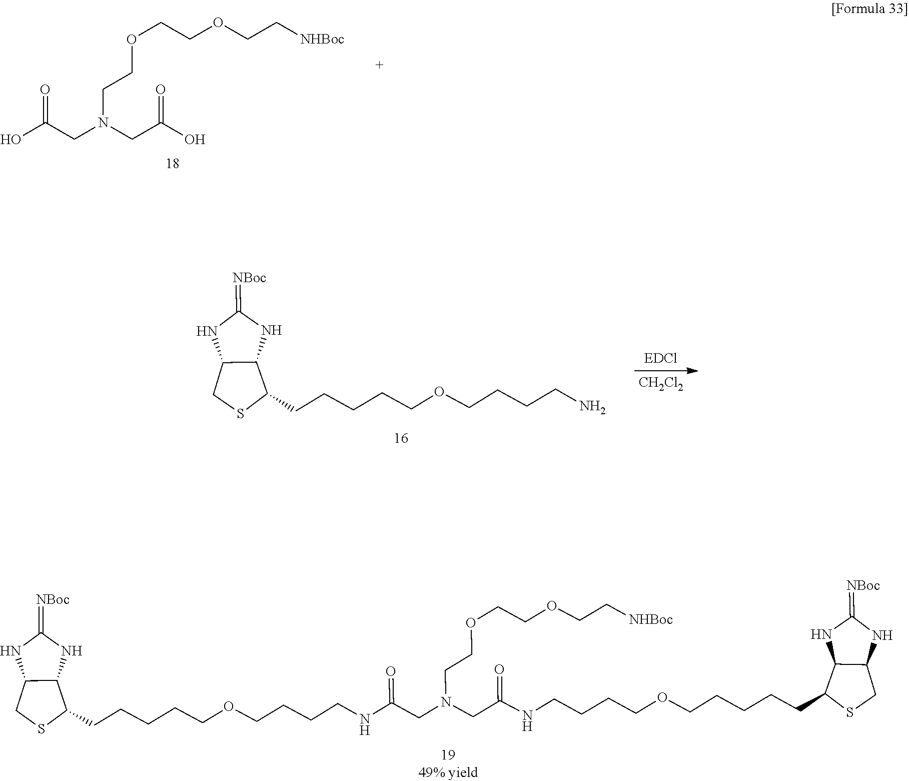

The Compound 16 (12.9 mg, 32.2 μmol) and 1-ethyl-3-(3-dimethylaminopropyl) carbodiimide hydrochloride (6.9 mg, 36 μmol) were added to a solution of the compound 18 (6.4 mg, 17.5 μmol) in dichloromethane (400 μl) at room temperature. The obtained mixture was stirred in an argon atmosphere at room temperature for 36 hours, and the solvent was then removed under reduced pressure. The obtained crude product was purified by silica gel chromatography (amino silica, chloroform/methanol=200:1→50:1→30:1→20:1→10:1→5:1→3:1) to obtain the target compound 19 (9.6 mg, a yield of 49%).

¹H NMR (400 MHz, CD₃OD) δ:1.34-1.50 (m, 35H), 1.51-1.64 (m, 14H), 1.72-1.82 (m, 2H), 2.81 (d, J=12.6 Hz, 2H), 2.97 (dd, J=4.9 Hz, 12.6 Hz, 2H), 3.18-3.30 (m, 10H), 3.39-3.65 (m, 20H), 4.41 (dd, J=4.5 Hz, 8.1 Hz, 2H), 4.63 (dd, J=4.5 Hz, 8.1 Hz, 2H). LRMS (ESI): m/z 565 [M+2H]²⁺.

### 2,2′-((2-(2-(2-Aminoethoxy)ethoxy)ethyl)azanediyl)bis(N-(4-((5-((3aS,4S,6aR)-2-iminohexahydro-1H-thieno[3,4-d]imidazol-4-yl)pentyl)oxy)butyl)acetamide) 2,2,2-trifluoroacetate

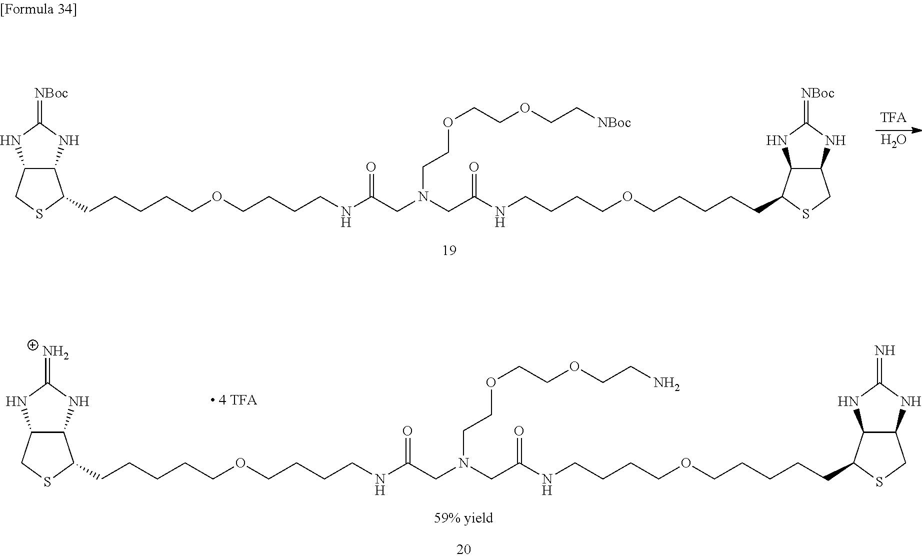

Trifluoroacetic acid (400 μl) was added to an aqueous solution (200 μl) of the compound 19 (5.2 mg, 4.6 μmol) at room temperature. The obtained mixture was stirred in an argon atmosphere at room temperature for 18 hours, and the solvent was then removed under reduced pressure. The obtained crude product was purified by reverse phase HPLC (gradient: 0% for 5 min; 0-100% for 90 min CH₃CN in 0.1% CF₃COOH aqueous solution, YMC-Triart C18, flow rate=3.5 ml/min) to obtain the target compound 20 (3.5 mg, a yield of 59%).

¹H NMR (500 MHz, CD₃OD) δ:1.34-1.50 (m, 8H), 1.52-1.63 (m, 14H), 1.71-1.81 (m, 2H), 2.82 (d, J=13.2 Hz, 2H), 2.99 (dd, J=4.6 Hz, 13.2 Hz, 2H), 3.14 (t, J=4.6 Hz, 4H), 3.20-3.30 (m, 6H), 3.43 (t, J=6.3 Hz, 8H), 3.60-3.74 (m, 12H), 4.53 (dd, J=4.0 Hz, 8.0 Hz, 2H), 4.72 (dd, J=4.6 Hz, 8.0 Hz, 2H). LRMS (ESI): m/z 415 [M+2H]²⁺.

### 2-((4-((5-((3aS,4S,6aR)-2-Iminiohexahydro-1H-thieno[3,4-d]imidazol-4-yl)pentyl)oxy)butyl)amino)-N-(2-((4-((5-((3aS,4S,6aR)-2-iminiohexahydro-1H-thieno[3,4-d]imidazol-4-yl)pentyl)oxy)butyl)amino)-2-oxoethyl)-2-oxo-N-(2-(2-(2-(3-(trimethylstannyl)benzamido)ethoxy)ethoxy)ethyl)ethan-1-aminium formate

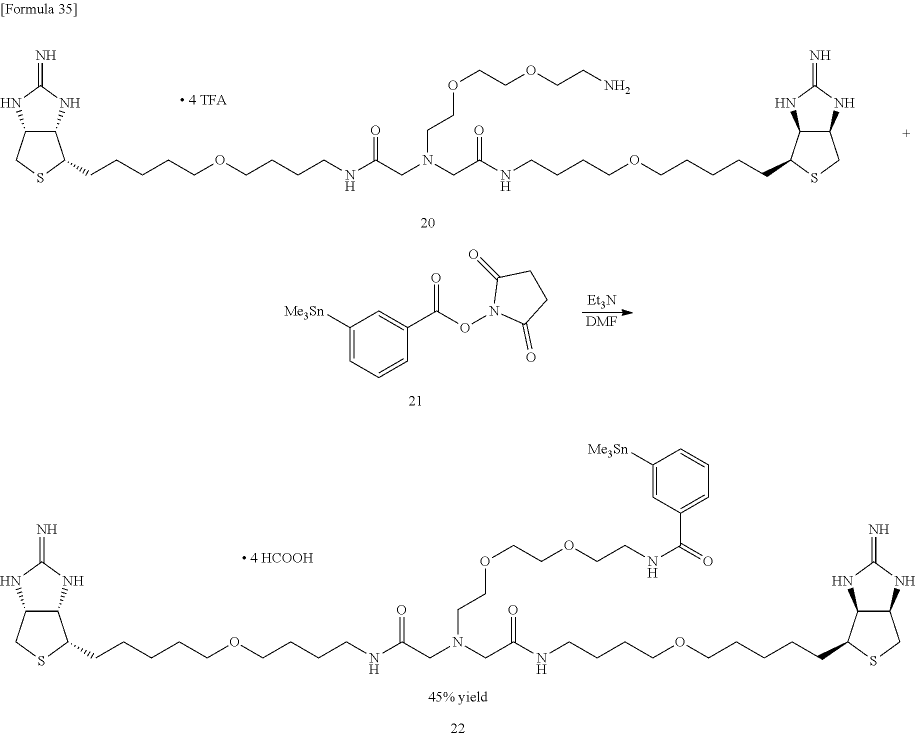

Compound 21 (1.1 mg, 2.99 μmol) and triethylamine (5.7 μl, 40.8 μmol) were added to a solution of the compound 20 (3.5 mg, 2.72 μmol) in DMF (300 μl) at room temperature. The obtained mixture was stirred in an argon atmosphere at room temperature for 3 hours, and the solvent was then removed under reduced pressure. The obtained crude product was purified by reverse phase HPLC (gradient: 0% for 5 min; 2-100% for 90 min CH₃CN in 0.1% HCOOH aqueous solution, retention time: 42.0 min, YMC-Triart C18, flow rate=3.5 ml/min) to obtain the target compound 22 (1.5 mg, a yield of 45%). LRMS (ESI): m/z 366 [M+3H]³⁺.

### 2,2′-((2-(2-(2-(3-Iodobenzamido)ethoxy)ethoxy)ethyl)azanediyl)bis(N-(4-((5-((3aS,4S,6aR)-2-iminohexahydro-1H-thieno[3,4-d]imidazol-4-yl)pentyl)oxy)butyl)acetamide) 2,2,2-trifluoroacetate

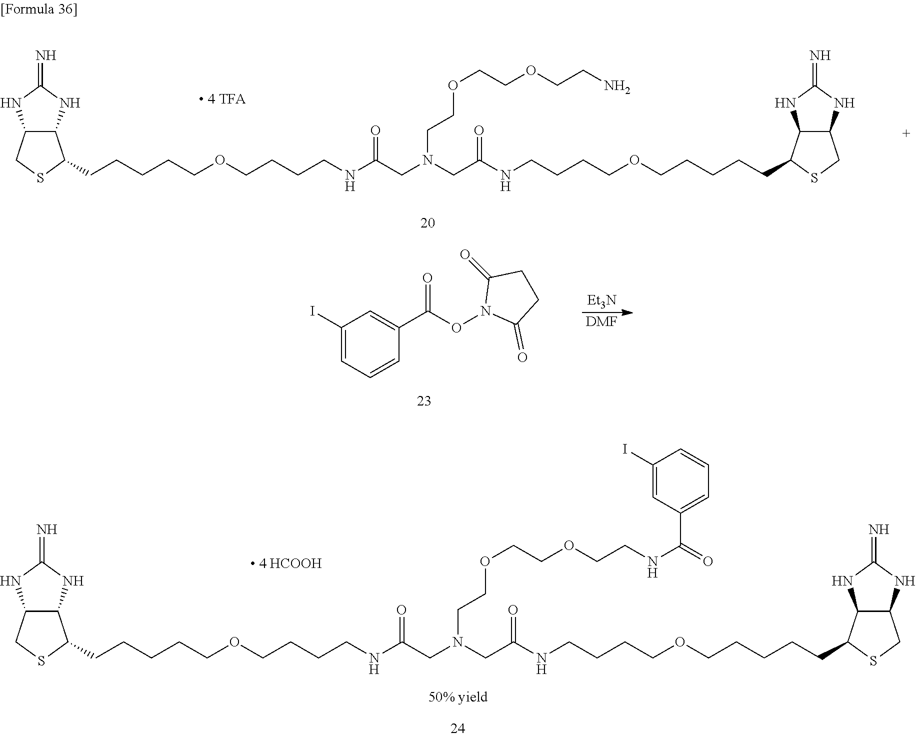

Compound 23 (0.7 mg, 2.06 μmol) dissolved in DMF (100 μl) and triethylamine (4 μl, 28.3 μmol) were added to a solution of the bisiminobiotin 20 (2.4 mg, 1.87 μmol) in DMF (150 μl) at room temperature. The obtained mixture was stirred in an argon atmosphere at room temperature for 2 hours, and the solvent was then removed under reduced pressure. The obtained crude product was purified by reverse phase HPLC (gradient: 0% for 5 min; 2-100% for 90 min CH₃CN in 0.1% CF₃COOH aqueous solution, retention time: 47.6 min, YMC-Triart C18, flow rate=3.5 ml/min) to obtain the target compound 24 (1.3 mg, a yield of 50%). LRMS (ESI): m/z 354 [M+3H]³⁺. The target compound 24 is also referred to as “Psyche P-iodine.”

### 2,2′-((2-(2-(2-(3-Iodobenzamido)ethoxy)ethoxy)ethyl)azanediyl)bis(N-(4-((5-((3aS,4S,6aR)-2-iminohexahydro-1H-thieno[3,4-d]imidazol-4-yl)pentyl)oxy)butyl)acetamide) 2,2,2-trifluoroacetate

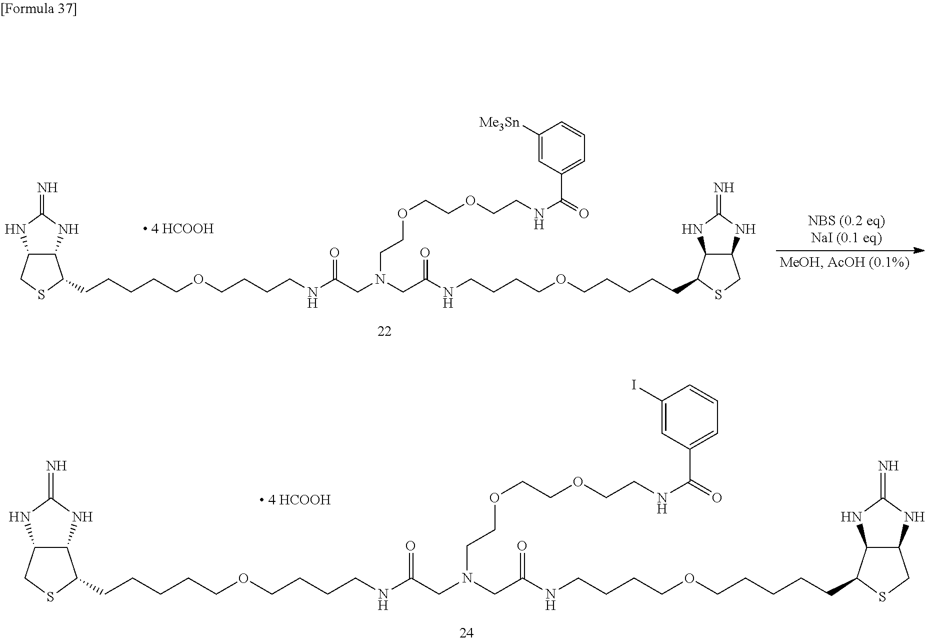

Sodium iodide (0.00121 mg, 0.081 μmol) dissolved in methanol (100 μl) was added to a solution of N-Bromo-succinimide (0.00288 mg, 0.162 μmol) in a mixture of methanol (610 μl) and acetic acid (0.81 μl), and the obtained mixture was then stirred at room temperature for 5 minutes. Thereafter, the bisiminobiotin 22 (0.1 mg, 0.081 μmol) dissolved in methanol (100 μl) was added to the reaction solution, and the thus obtained mixture was then stirred at room temperature for 10 minutes. Thereafter, the reaction solution was subjected to LC-MS analysis, and as a result, the reaction solution was identical to the preparation (24) synthesized by the above-described method, in terms of the retention time and the measured mass. The target compound 24 is also referred to as “Psyche P-iodine.”

### <Production of CEA-V2122>

V2122 is a mutant streptavidin described in Example 3 of International Publication WO2015/125820 (SEQ ID NO: 4 shown in International Publication WO2015/125820). The amino acid sequence of V2122 (a sequence having 6×His tag at the C-terminus) is as set forth in SEQ ID NO: 1 in the sequence listing.

scFv-V2122 is prepared by binding a single-chain antibody (scFv) against CEACAM5 with the above-described V2122. This scFv-type anti-CEACAM5 antibody is an scFv sequence described in a patent document U.S. Pat. No. 7,626,011B2. The amino acid sequence of the scFv-type anti-CEACAM5 antibody is as set forth in SEQ ID NO: 2 in the sequence listing. In addition, the amino acid sequence of CEA-V2122 prepared by binding the scFv-type anti-CEACAM5 antibody with V2122 via an amino acid linker (GGGGSGGGG) (SEQ ID NO: 11) is as set forth in SEQ ID NO: 3 in the sequence listing.

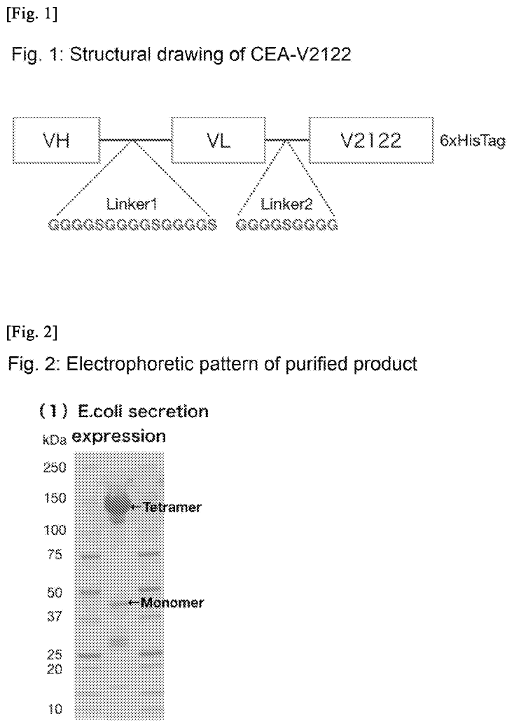

For the expression of a CEA-V2122 fusion protein, the DNA codon of a CEA-V2122 gene sequence, in which a pelB signal for secretion and expression in Escherichia coli had been incorporated into the N-terminus and a 6×His-Tag sequence had been incorporated into the C-terminus, was optimized for Escherichia coli, thereby synthesizing an artificial gene. This amino acid sequence is as set forth in SEQ ID NO: 4 in the sequence listing, and the DNA sequence is as set forth in SEQ ID NO: 5 in the sequence listing. Moreover, an outline of a domain structure is shown in FIG. 1.

As a specific protein expression vector, a vector prepared by incorporating a chaperone skp gene into MCS2 of a pETDuetl vector was used. Regarding the skp gene, the DNA codon was optimized for Escherichia coli based on the amino acid sequence as set forth in SEQ ID NO: 6 in the sequence listing, thereby synthesizing an artificial gene. The synthesized skp gene was amplified by PCR, using the primers (AAGGAGATATACATATGGATAAAATTGCCATTGTTAATAT (SEQ ID NO: 12) and TTGAGATCTGCCATATGTTATTTCACTTGTTTCAGAACG (SEQ ID NO: 13)), and the amplified gene was then cloned into MCS2 of the pETDue1 vector linearized with the restriction enzyme NdeI, using In-Fusion HD Cloning Kit, so as to obtain pETDuet_skp. Subsequently, the CEA-V2122 gene was incorporated into MCS1 of pETDuet_skp. Specifically, the artificially synthesized CEA-V2122 gene was amplified by PCR, using the primers (AGAAGGAGATATACCATGAAATATCTGCTGCCGAC (SEQ ID NO: 14) and CGCCGAGCTCGAATTTTAATGATGGTGATGATGATG (SEQ ID NO: 15)). Moreover, pETDuet_skp was linearized by PCR, using the primers (GGTATATCTCCTTCTTAAAGTTAAAC (SEQ ID NO: 16) and AATTCGAGCTCGGCGCGCCTGCAG (SEQ ID NO: 17)). The CEA-V2122 amplified by PCR and the linearized pETDuet_skp were subjected to cloning using In-Fusion HD Cloning Kit. The cloned vector was confirmed by sequencing, in terms of a gene sequence incorporated therein, and it was referred to as pETDuet_CEA-V2122_skp.

For the expression of the protein, pETDuet_CEA-V2122_skp was transformed into BL21(DE3) (Nippon Gene Co., Ltd.), which was then pre-cultured in 2×YT medium (SIGMA-ADLRICH) at 37° C. overnight. The medium used in the pre-culture was added to a new medium to 100-fold dilution, and culture was then carried out at 37° C. until OD (600 nm)=0.5 to 2.0. Subsequently, IPTG was added to the culture to a final concentration of 0.5 mM, and the obtained mixture was then cultured at 37° C. for 4 hours. Thereafter, a culture supernatant was recovered and was then preserved at 4° C.

The CEA-V2122 protein was roughly purified according to a batch method utilizing 6×His-Tag added to the C-terminus. Specifically, cOmplete His-Tag Purification Resin equilibrated with buffer A (50 mM Tris-HCl, 0.2 M NaCl, 1 mM EDTA, and 5 mM Imidazole; pH 8.0) was added to the culture supernatant preserved at 4° C. The obtained mixture was stirred from 2 hours to overnight at 4° C., so that the protein was allowed to bind to the resin. Subsequently, the resin was recovered into a column, and a 20 column volume of washing operation was performed with buffer A. Thereafter, a roughly purified product of CEA-V2122 was recovered by elution with buffer B (50 mM Tris-HCl, 0.2 M NaCl, 1 mM EDTA, and 400 mM Imidazole; pH 8.0).

Subsequently, the roughly purified product was purified using a Protein L column. Specifically, 1 mL of Capto L (GE Healthcare Life Sciences) was filled into a PD-10 column and was then equilibrated with 10 column volume of PBS, and the aforementioned roughly purified product was then applied thereto. Thereafter, the resultant was washed with 10 column volume of PBS, was then eluted with 10 mM glycine hydrochloride (pH 2.0), and was then subjected to centrifugal concentration using Vivaspin Turbo 15 (MWCO 100,000). Moreover, using PD-10 (GE Healthcare Life Science), the buffer was replaced with PBS, and centrifugal concentration was further carried out using Vivaspin Turbo 4 (MWCO 100,000) to obtain a finally purified product. After completion of SDS-PAGE electrophoresis, the purity of tetramer CEA-V2122 was assayed by CBB staining. The results are shown in FIG. 2. As SDS-PAGE gel, Mini-PROTEAN TGX 4-15% (Bio-Rad) was used, and as a CBB staining solution, Bullet CBB Stain One (Ready To Use) (Nacalai Tesque, Inc.) was used.

From FIG. 2, it was confirmed that the purified CEA-V2122 comprises an approximately 150-kDa tetramer as a main component.

### <Production of Herceptin-V2122>

V2122 is a mutant streptavidin described in Example 3 of International Publication WO2015/125820 (SEQ ID NO: 4 shown in International Publication WO2015/125820). The amino acid sequence of V2122 is as set forth in SEQ ID NO: 1 in the sequence listing.

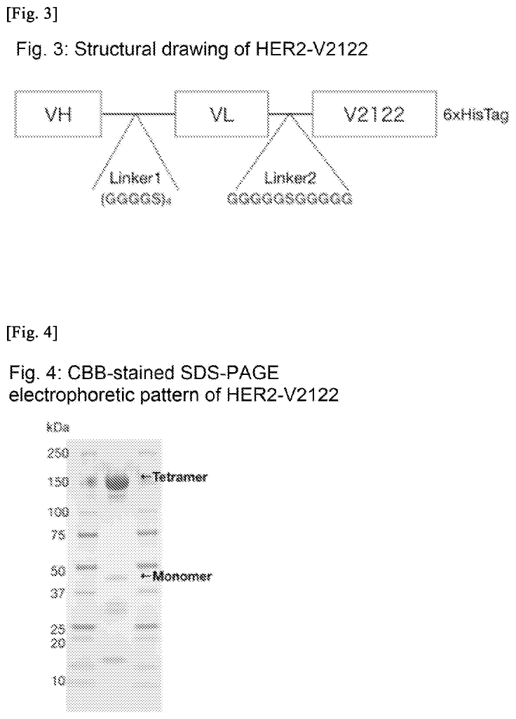

scFv-V2122 is prepared by binding a single-chain antibody (scFv) against HER2 (ERBB2) with the above-described V2122. This scFv-type anti-HER2 antibody is an scFv sequence described in Zhang H, et al., Therapeutic potential of an anti-HER2 single chain antibody-DM1 conjugates for the treatment of HER2-positive cancer. Signal Transduct Target Ther. 2017 May 19; 2:17015. doi: 10.1038/sigtrans. 2017.15. The amino acid sequence of the scFv-type anti-HER2 antibody is as set forth in SEQ ID NO: 7 in the sequence listing. In addition, the structural drawing of HER2-V2122 prepared by binding the scFv-type anti-HER2 antibody with V2122 via an amino acid linker (GGGGGSGGGGG) (SEQ ID NO: 18) is shown in FIG. 3, and the amino acid sequence of HER2-V2122 is as set forth in SEQ ID NO: 8 in the sequence listing.

For the expression of a HER2-V2122 fusion protein, the DNA codon of a HER2-V2122 gene sequence, in which a pelB signal for secretion and expression in Escherichia coli had been incorporated into the N-terminus and a 6×His-Tag sequence had been incorporated into the C-terminus, was optimized for Escherichia coli, thereby synthesizing an artificial gene. This amino acid sequence is as set forth in SEQ ID NO: 9 in the sequence listing, and the DNA sequence is as set forth in SEQ ID NO: 10 in the sequence listing.

As a specific protein expression vector, a vector prepared by incorporating a chaperone skp gene into MCS2 of a pETDuetl vector was used. Regarding the skp gene, the DNA codon was optimized for Escherichia coli based on the amino acid sequence as set forth in SEQ ID NO: 6 in the sequence listing, thereby synthesizing an artificial gene. The synthesized skp gene was amplified by PCR, using the primers (AAGGAGATATACATATGGATAAAATTGCCATTGTTAATAT (SEQ ID NO: 12) and TTGAGATCTGCCATATGTTATTTCACTTGTTTCAGAACG (SEQ ID NO: 13)), and the amplified gene was then cloned into MCS2 of the pETDue1 vector linearized with the restriction enzyme NdeI, using In-Fusion HD Cloning Kit, so as to obtain pETDuet_skp. Subsequently, the HER2-V2122 gene was incorporated into MCS1 of pETDuet_skp. Specifically, the artificially synthesized HER2-V2122 gene was amplified by PCR, using the primers (AGAAGGAGATATACCATGAAATATCTGCTGCCGAC (SEQ ID NO: 14) and CGCCGAGCTCGAATTTTAATGATGGTGATGATGATG (SEQ ID NO: 15)). Moreover, pETDuet_skp was linearized by PCR, using the primers (GGTATATCTCCTTCTTAAAGTTAAAC (SEQ ID NO: 16) and AATTCGAGCTCGGCGCGCCTGCAG (SEQ ID NO: 17)). The CEA-V2122 amplified by PCR and the linearized pETDuet_skp were subjected to cloning using In-Fusion HD Cloning Kit. The cloned vector was confirmed by sequencing, in terms of a gene sequence incorporated therein, and it was referred to as pETDuet_HER2-V2122_skp.

For the expression of the protein, pETDuet_HER2-V2122_skp was transformed into BL21(DE3) (Nippon Gene Co., Ltd.), which was then pre-cultured in 2×YT medium (SIGMA-ADLRICH) at 37° C. overnight. The medium used in the pre-culture was added to a new medium to 100-fold dilution, and culture was then carried out at 37° C. until OD (600 nm)=0.5 to 2.0. Subsequently, IPTG was added to the culture to a final concentration of 0.5 mM, and the obtained mixture was then cultured at 37° C. for 4 hours. Thereafter, a culture supernatant was recovered and was then preserved at 4° C.

The HER2-V2122 protein was roughly purified according to a batch method utilizing 6×His-Tag added to the C-terminus. Specifically, cOmplete His-Tag Purification Resin equilibrated with buffer A (50 mM Tris-HCl, 0.2 M NaCl, 1 mM EDTA, and 5 mM Imidazole; pH 8.0) was added to the culture supernatant preserved at 4° C. The obtained mixture was stirred from 2 hours to overnight at 4° C., so that the protein was allowed to bind to the resin. Subsequently, the resin was recovered into a column, and a 20 column volume of washing operation was performed with buffer A. Thereafter, a roughly purified product of HER2-V2122 was recovered by elution with buffer B (50 mM Tris-HCl, 0.2 M NaCl, 1 mM EDTA, and 400 mM Imidazole; pH 8.0).

Subsequently, the roughly purified product was purified using a Protein L column. Specifically, 1 mL of Capto L (GE Healthcare) was filled into a PD-10 column and was then equilibrated with 10 column volume of PBS, and the aforementioned roughly purified product was then applied thereto. Thereafter, the resultant was washed with 10 column volume of PBS, was then eluted with 10 mM glycine hydrochloride (pH 2.0), and was then subjected to centrifugal concentration using Vivaspin Turbo 15 (MWCO 100,000). Moreover, using PD-10 (GE Healthcare), the buffer was replaced with PBS, and centrifugal concentration was further carried out using Vivaspin Turbo 4 (MWCO 100,000) to obtain a finally purified product. The purified product was subjected to CBB staining, and the purity of tetramer HER2-V2122 was assayed. The results are shown in FIG. 4. As SDS-PAGE gel, Mini-PROTEAN TGX 4-15% (Bio-Rad) was used, and as a CBB staining solution, Bullet CBB Stain One (Ready To Use) (Nacalai Tesque, Inc.) was used.

From FIG. 4, it was confirmed that the purified HER2-V2122 comprises an approximately 150-kDa tetramer as a main component.

### Test Example 1: Analysis of Affinity of CEA-V2122 for Psyche B-Iodine and for Psyche P-Iodine

The data regarding the affinity of CEA-V2122 for Psyche B-iodine and for Psyche P-iodine were obtained in accordance with the description of Japanese Patent Application 2018-247363. Briefly stating, using an amine coupling kit (GE Healthcare Life Sciences), Sensor Chip CM5 (GE Healthcare Life Science) was set to be a target value that was 5000 RU, and the purified CEA-V2122 was immobilized. With regard to the concentration of the analyte, 5 types of two-fold serial dilutions from 1E-08 M to 6.25E-10 M were used. Biacore T200 (GE Healthcare Life Sciences) was used, the measurement temperature was set to be 25° C., and kinetics data were then obtained according to Single-Cycle Kinetics analysis. Using Biacore T200 Evaluation Software, version 2.0 (GE Healthcare Life Sciences), the obtained data were subjected to curve fitting in a Bivalent Analyze mode, and the evaluation value of each parameter was obtained. The dissociation constant K_{D} is obtained according to Kd/Ka=K_{D}. The results are shown in Table 1. From the results, it was confirmed that Psyche B-iodine and Psyche P-iodine form a stable bond with CEA-V2122.

TABLE 1

| | Ka (Ms⁻¹) | Kd (s⁻¹) | K_{D} (M) |
|---|---|---|---|
| Psyche B-iodine | 2.9E+04 | 6.4E−07 | 2.2E−11 |
| Psyche P-iodine | 6.8E+03 | 3.1E−05 | 4.5E−09 |

### Test Example 2: Confirmation of Binding Conjugate of Astatine-Labeled Psyche B and Herceptin-Cupid by Using Magnetic Beads

In order to confirm whether or not astatine-labeled Psyche B has binding ability to Cupid, a binding assay was carried out using Herceptin-Cupid and Ni-NTA magnetic beads (manufactured by Thermo) that bind to a 6×His-tag added to Herceptin-Cupid. Psyche B-At211 used herein was prepared according to Example 1(4), whereas Herceptin-Cupid was prepared according to the method described in Japanese Patent Application 2018-247363, as described above. The specific assay method is as follows.

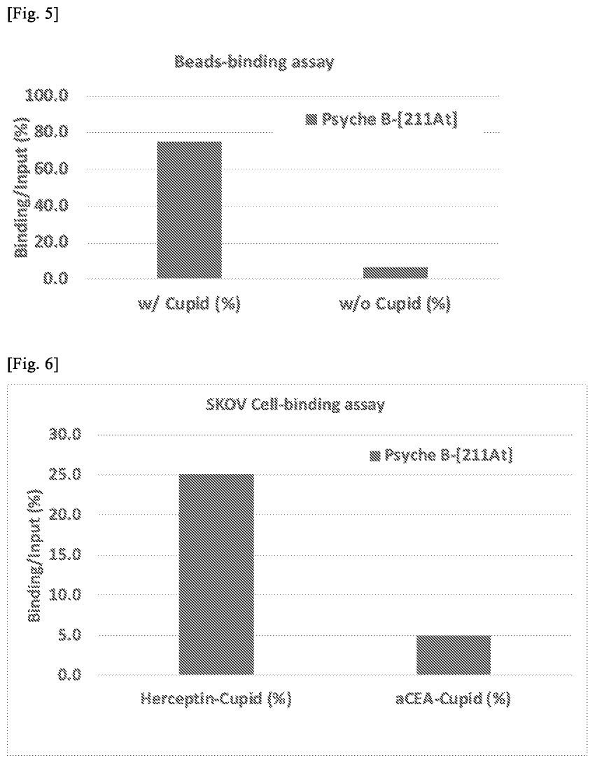

That is, 4 μL of Herceptin-V2122 solution or PBS used as a control was dispensed into a 1.5-mL tube. Into the tube, 10 μL (corresponding to 5.8 kBq) of Psyche-At211 was added, and the obtained mixture was then incubated at room temperature for 5 minutes. Subsequently, 10 μL of Ni-NTA magnetic beads equilibrated with PBS were added to the reaction mixture, and the thus obtained mixture was then incubated at room temperature for 5 minutes. Thereafter, the beads were precipitated and immobilized by using a magnet, and a supernatant was then recovered. The supernatant was washed with 100 μL of PBS twice, and the radiation dose was then counted using a gamma counter. The results are shown in the graph of FIG. 5. It was confirmed that Psyche-At211 was precipitated in a Herceptin-V2122 presence-dependent manner.

### Test Example 3: Confirmation of Binding Conjugate of Astatine-Labeled Psyche B and Herceptin-Cupid by Using Ovarian Cancer Cells SKOV3

Whether or not astatine-labeled Psyche B has binding ability to Herceptin-V2122, which had previously been bound to HER2-positive SKOV3 cells, was confirmed. As a negative control, CEA-V2122 was used. Psyche B-At211 used herein was prepared according to Example 1(4), whereas Herceptin-V2122 and CEA-V2122 were prepared according to the method described in Japanese Patent Application 2018-247363, as described above. The specific assay method is as follows.

Four 1.5-mL tubes were prepared, and the cells were then dispersed into each tube to a cell density of 0.875×10⁶ cells/tube. Herceptin-V2122 was added to two tubes to result in N=2, whereas CEA-V2122 was added to the remaining two tubes. Subsequently, incubation was carried out on ice for 1 hour, and astatine-labeled Psyche B was then added to the resultant to 10 kBq/tube. Thereafter, the obtained mixture was incubated at room temperature for 30 minutes, while shaking. Finally, the cells were precipitated by centrifugation, and a supernatant was then recovered. The supernatant was washed with PBS twice, and the radiation dose was then counted using a gamma counter. The results are shown in the graph of FIG. 6. The measurement results were Herceptin-V2122-dependent, and it was confirmed that Psyche B-At212 bound to the SKOV3 cells via Herceptin-V2122.

SEQ ID NO: 1
AEAGITGTWSDQLGDTFIVTAGADGALTGTYENAVGGAESRYVLTGRYD
SAPATDGSGTALGWTVAWKNNSKNAHSATTWSGQYVGGADAKINTQWLL
TSGTTNANAWKSTLVGHDTFTKVKPSAASHHHHHH
(sm3E-scFv sequence)
SEQ ID NO: 2
QVKLEQSGAEVVKPGASVKLSCKASGFNIKDSYMHWLRQGPGQRLEWIG
WIDPENGDTEYAPKFQGKATFTTDTSANTAYLGLSSLRPEDTAVYYCNE
GTPTGPYYFDYWGQGTLVTVSSGGGGSGGGGSGGGGSENVLTQSPSSMS
VSVGDRVNIACSASSSVPYMHWLQQKPGKSPKLLIYLTSNLASGVPSRF
SGSGSGTDYSLTISSVQPEDAATYYCQQRSSYPLTFGGGTKLEIK
SEQ ID NO: 3
QVKLEQSGAEVVKPGASVKLSCKASGFNIKDSYMHWLRQGPGQRLEWIG
WIDPENGDTEYAPKFQGKATFTTDTSANTAYLGLSSLRPEDTAVYYCNE
GTPTGPYYFDYWGQGTLVTVSSGGGGSGGGGSGGGGSENVLTQSPSSMS
VSVGDRVNIACSASSSVPYMHWLQQKPGKSPKLLIYLTSNLASGVPSRF
SGSGSGTDYSLTISSVQPEDAATYYCQQRSSYPLTFGGGTKLEIKGGGG
SGGGGAEAGITGTWSDQLGDTFIVTAGADGALTGTYENAVGGAESRYVL
TGRYDSAPATDGSGTALGWTVAWKNNSKNAHSATTWSGQYVGGADAKIN
TQWLLTSGTTNANAWKSTLVGHDTFTKVKPSAASHHHHHH
SEQ ID NO: 4
MKYLLPTAAAGLLLLAAQPAMAQVKLEQSGAEVVKPGASVKLSCKASGF
NIKDSYMHWLRQGPGQRLEWIGWIDPENGDTEYAPKFQGKATFTTDTSA
NTAYLGLSSLRPEDTAVYYCNEGTPTGPYYFDYWGQGTLVTVSSGGGGS
GGGGSGGGGSENVLTQSPSSMSVSVGDRVNIACSASSSVPYMHWLQQKP
GKSPKLLIYLTSNLASGVPSRFSGSGSGTDYSLTISSVQPEDAATYYCQ
QRSSYPLTFGGGTKLEIKGGGGSGGGGAEAGITGTWSDQLGDTFIVTAG
ADGALTGTYENAVGGAESRYVLTGRYDSAPATDGSGTALGWTVAWKNNS
KNAHSATTWSGQYVGGADAKINTQWLLTSGTTNANAWKSTLVGHDTFTK
VKPSAASHHHHHH
SEQ ID NO: 5
ATGAAATATCTGCTGCCGACCGCAGCAGCGGGTCTGCTGCTGCTGGCAG
CACAGCCTGCAATGGCACAGGTTAAACTGGAACAGAGCGGTGCCGAAGT
TGTTAAACCGGGTGCAAGCGTTAAACTGAGCTGTAAAGCAAGCGGCTTT
AACATCAAAGATAGCTATATGCATTGGCTGCGTCAGGGTCCGGGTCAGC
GTCTGGAATGGATTGGTTGGATTGATCCGGAAAATGGTGATACCGAATA
TGCACCGAAATTTCAGGGTAAAGCAACCTTTACCACCGATACCAGCGCA
AATACCGCATATCTGGGTCTGAGCAGCCTGCGTCCGGAAGATACCGCAG
TGTATTATTGTAATGAAGGCACCCCGACCGGTCCGTATTATTTCGATTA
TTGGGGTCAGGGCACCCTGGTTACCGTTAGCAGCGGTGGTGGTGGTAGT
GGTGGCGGTGGTTCAGGCGGTGGCGGTAGCGAAAATGTTCTGACCCAGA
GCCCGAGCAGCATGAGCGTTAGCGTTGGTGATCGTGTTAATATTGCATG
TAGCGCAAGCAGCAGCGTTCCGTACATGCACTGGCTGCAGCAGAAACCG
GGTAAAAGCCCGAAACTGCTGATTTATCTGACCAGCAATCTGGCAAGCG
GTGTTCCGAGCCGTTTTAGCGGTAGCGGTAGTGGCACCGATTATAGCCT
GACCATTAGCAGCGTGCAGCCTGAAGATGCAGCAACCTATTATTGTCAG
CAGCGTAGCAGTTATCCGCTGACCTTTGGTGGTGGCACCAAACTGGAAA
TTAAAGGGGGTGGTGGCTCAGGTGGCGGAGGTGCAGAAGCAGGTATTAC
CGGTACATGGTCAGATCAGCTGGGTGATACCTTTATTGTTACCGCAGGC
GCAGATGGTGCACTGACCGGCACCTATGAAAATGCAGTTGGTGGTGCAG
AAAGCCGTTATGTGCTGACCGGTCGTTATGATAGCGCACCGGCAACCGA
TGGTAGCGGCACCGCACTGGGTTGGACCGTTGCATGGAAAAATAACAGC
AAAAATGCACATAGCGCAACCACCTGGTCAGGTCAGTATGTGGGTGGTG
CCGATGCCAAAATTAACACCCAGTGGCTGCTGACCAGCGGTACAACCAA
TGCAAATGCCTGGAAAAGTACCCTGGTTGGTCATGATACATTCACCAAA
GTTAAACCGAGCGCAGCAAGCCATCATCATCACCATCATTAA
SEQ ID NO: 6
MDKIAIVNMGSLFQQVAQKTGVSNTLENEFKGRASELQRMETDLQAKMK
KLQSMKAGSDRTKLEKDVMAQRQTFAQKAQAFEQDRARRSNEERGKLVT
RIQTAVKSVANSQDIDLVVDANAVAYNSSDVKDITADVLKQVK
SEQ ID NO: 7
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSGGGGSGGGGSGGGGSGGGGSDIQMTQS
PSSLSASVGDRVTITCRASQDVNTAVAWYQQKPGKAPKLLIYSASFLYS
GVPSRFSGSRSGTDFTLTISSLQPEDFATYYCQQHYTTPPTFGQGTKVE
IK
SEQ ID NO: 8
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSGGGGSGGGGSGGGGSGGGGSDIQMTQS
PSSLSASVGDRVTITCRASQDVNTAVAWYQQKPGKAPKLLIYSASFLYS
GVPSRFSGSRSGTDFTLTISSLQPEDFATYYCQQHYTTPPTFGQGTKVE
IKGGGGGSGGGGGAEAGITGTWSDQLGDTFIVTAGADGALTGTYENAVG
GAESRYVLTGRYDSAPATDGSGTALGWTVAWKNNSKNAHSATTWSGQYV
GGADAKINTQWLLTSGTTNANAWKSTLVGHDTFTKVKPSAASHHHHHH
SEQ ID NO: 9
MKYLLPTAAAGLLLLAAQPAMAEVQLVESGGGLVQPGGSLRLSCAASGF
NIKDTYIHWVRQAPGKGLEWVARIYPTNGYTRYADSVKGRFTISADTSK
NTAYLQMNSLRAEDTAVYYCSRWGGDGFYAMDYWGQGTLVTVSSGGGGS
GGGGSGGGGSGGGGSDIQMTQSPSSLSASVGDRVTITCRASQDVNTAVA
WYQQKPGKAPKLLIYSASFLYSGVPSRFSGSRSGTDFTLTISSLQPEDF
ATYYCQQHYTTPPTFGQGTKVEIKGGGGGSGGGGGAEAGITGTWSDQLG
DTFIVTAGADGALTGTYENAVGGAESRYVLTGRYDSAPATDGSGTALGW
TVAWKNNSKNAHSATTWSGQYVGGADAKINTQWLLTSGTTNANAWKSTL
VGHDTFTKVKPSAASHHHHHH
SEQ ID NO: 10
ATGAAATATCTGCTGCCGACCGCAGCAGCGGGTCTGCTGCTGCTGGCAG
CACAGCCTGCAATGGCAGAAGTTCAGCTGGTTGAAAGCGGTGGTGGTCT
GGTTCAGCCTGGTGGTAGCCTGCGTCTGAGCTGTGCAGCAAGCGGTTTT
AACATTAAAGATACCTATATTCATTGGGTGCGTCAGGCACCTGGTAAAG
GTCTGGAATGGGTTGCACGTATTTATCCGACCAATGGTTATACCCGTTA
TGCCGATAGCGTTAAAGGTCGTTTTACCATTAGCGCAGATACCAGCAAA
AATACCGCATACCTGCAGATGAATAGTCTGCGTGCAGAGGATACCGCAG
TGTATTATTGTAGCCGTTGGGGTGGTGATGGTTTTTATGCAATGGATTA
TTGGGGTCAGGGCACCCTGGTTACCGTTAGCTCAGGTGGAGGCGGTTCC
GGTGGCGGAGGTTCCGGTGGAGGTGGCTCCGGTGGCGGAGGTTCCGATA
TTCAGATGACCCAGAGTCCGAGCAGCCTGAGCGCAAGCGTTGGTGATCG
TGTGACCATTACCTGTCGTGCAAGCCAGGATGTTAATACAGCAGTTGCA
TGGTATCAGCAGAAACCGGGTAAAGCACCGAAACTGCTGATTTATAGCG
CAAGCTTTCTGTATAGCGGTGTTCCGAGCCGTTTTAGCGGTAGCCGTAG
CGGCACCGATTTTACCCTGACCATTAGCAGCCTGCAGCCGGAAGATTTT
GCAACCTATTATTGTCAGCAGCATTACACCACACCGCCTACCTTTGGCC
AGGGCACCAAAGTTGAAATTAAAGGAGGTGGCGGTGGATCCGGCGGAGG
TGGCGGAGCAGAAGCAGGTATTACCGGTACATGGTCAGATCAGCTGGGT
GATACCTTTATTGTTACCGCAGGCGCAGATGGTGCACTGACCGGCACCT
ATGAAAATGCAGTTGGTGGTGCAGAAAGCCGTTATGTGCTGACCGGTCG
TTATGATAGCGCACCGGCAACCGATGGTAGCGGCACCGCACTGGGTTGG
ACCGTTGCATGGAAAAATAACAGCAAAAATGCACATAGCGCAACCACCT
GGTCAGGTCAGTATGTGGGTGGTGCCGATGCCAAAATTAACACCCAGTG
GCTGCTGACCAGCGGTACAACCAATGCAAATGCCTGGAAAAGTACCCTG
GTTGGTCATGATACATTCACCAAAGTTAAACCGAGCGCAGCAAGCCATC
ATCATCACCATCATTAA

1. A compound represented by the following formula (1) or a salt thereof: 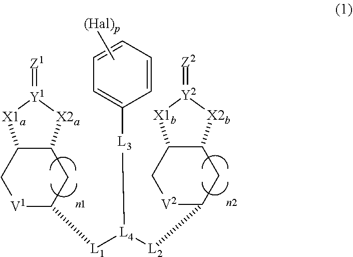
wherein
X1a, X1b, X2a and X2b each independently represent O or NH,
Y¹ and Y² each independently represent C or S,
Z¹ and Z² each independently represent O, S or NH,
V¹ and V² each independently represent S or S⁺—O⁻,
n1 and n2 each independently represent an integer of 0 or 1,
L₁, L₂, and L₃ each independently represent a divalent linking group,
L₄ represents a trivalent linking group,
Hal represents a halogen, and
p represents an integer of 1 to 5.

2. The compound according to claim 1, wherein the compound represented by the formula (1) is a compound represented by the following formula (1a) or the following formula (1b): 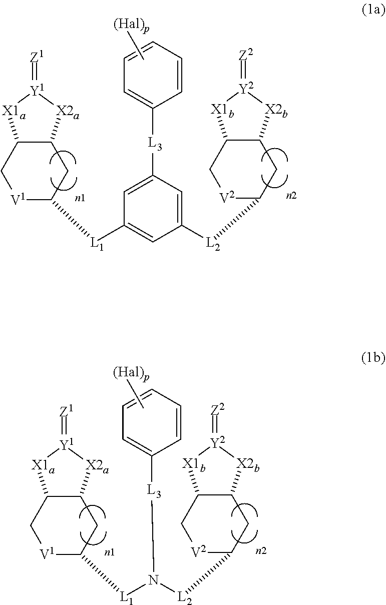
wherein each symbol is as defined in claim 1.

3. The compound or a salt thereof according to claim 1, which is represented by the following formula (2) or the following formula (3), wherein n1 and n2 are 0. 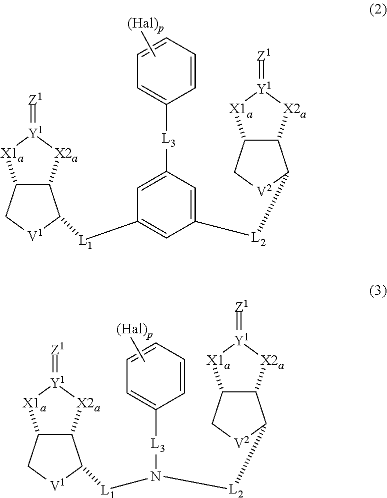
wherein each symbol is as defined in claim 1.

4. The compound or a salt thereof according to claim 1, wherein X1a, X1b, X2a and X2b represent NH, Y and Y² represent C, Z¹ and Z² represent NH, and V¹ and V² represent S.

5. The compound or a salt thereof according to claim 1, wherein L₁, L₂, and L₃ each independently represent a divalent linking group consisting of a combination of groups selected from —CONH—, —NHCO—, —COO—, —OCO—, —CO—, —O—, and an alkylene group containing 1 to 10 carbon atoms.

6. The compound or a salt thereof according to claim 1, wherein
L₁ represents —(CH₂)ₘ₁-L₁-(CH₂)ₘ₂-L₁₂-*,
L₂ represents *-L₁₃-(CH₂)ₘ₃-L₁₄-(CH₂)ₘ₄—, and
L₃ represents L₁₅-L₂₁-L₁₆-,
wherein L₁₁, L₁₂, L₁₃, L₁₄, L₁₅, and L₁₆ each independently represent —CONH—, —NHCO—, —COO—, —OCO—, —CO—, or —O—,
m₁, m₂, m₃, and m₄ each independently represent an integer from 1 to 10,
* represents a binding site with a benzene ring,
L21 represents —CH₂—(OCH₂)ₘ₅—, and
m₅ represents an integer from 1 to 10.

7. The compound or a salt thereof according to claim 1, wherein Hal represents I, ¹²⁵I, or ²¹¹At.

8. The compound or a salt thereof according to claim 1, wherein p is 1.

9. A compound represented by any one of the following formulae:

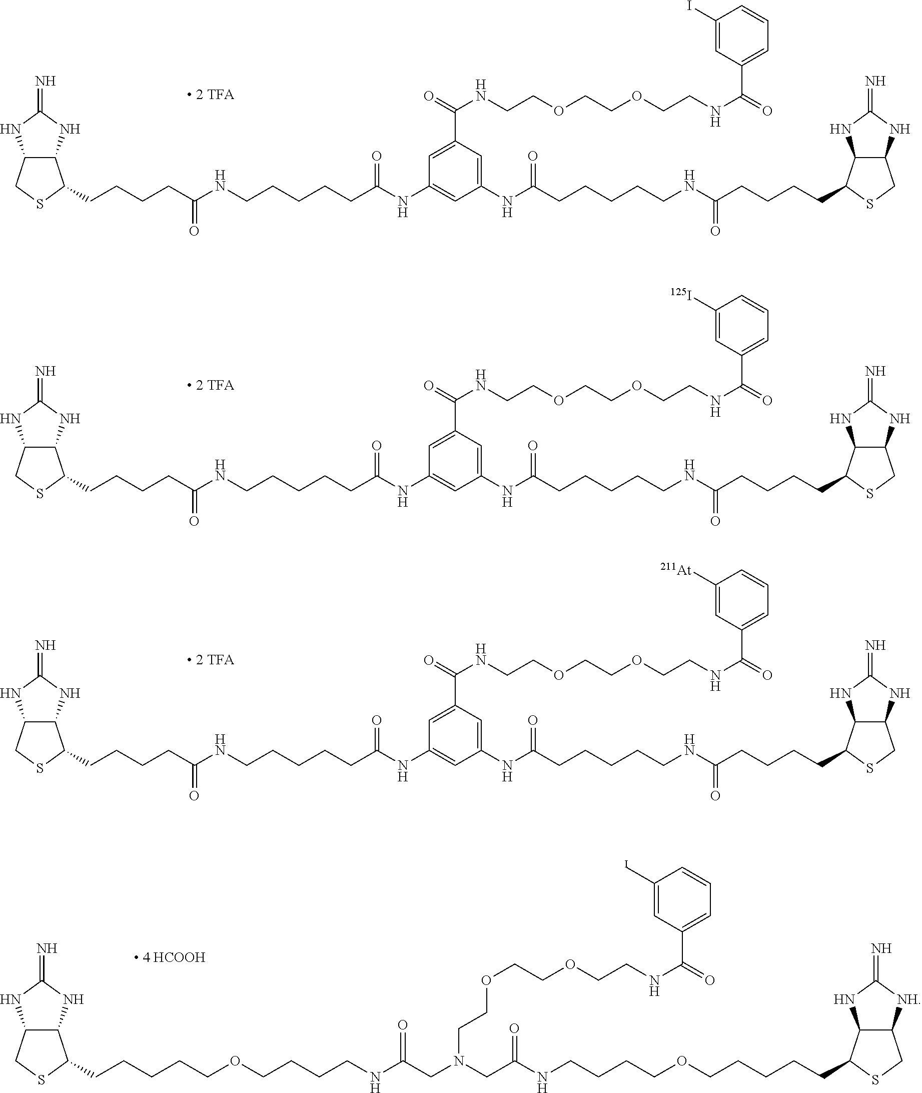

10. A kit for treatment or diagnosis, comprising: (1) the compound according to claim 1; and (b) a mutant streptavidin-molecular probe conjugate obtained by binding a molecular probe to a mutant streptavidin comprising the amino acid sequence as set forth in SEQ ID NO: 19.

11. A method for producing the compound or a salt thereof according to claim 1, comprising reacting a compound represented by the following formula (A) with sodium halide or halogen: 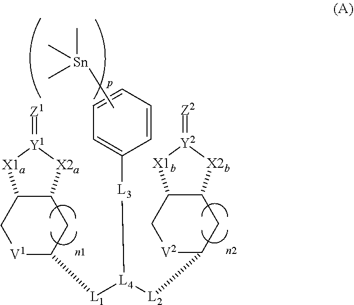
wherein each symbol is as defined in claim 1.

12. A method for producing the compound or a salt thereof according to claim 1, comprising reacting a compound represented by the following formula (B) with sodium halide or halogen: 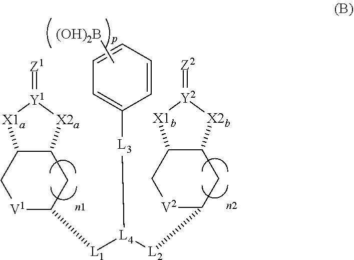
wherein each symbol is as defined in claim 1.


SEQUENCE LISTING (19 sequences)

SEQ ID NO: 1 - protein, 133 aa, Artificial Sequence
  Description of Artificial Sequence Recombinant protein
AEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 2 - protein, 241 aa, Artificial Sequence
  Description of Artificial Sequence Recombinant protein
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 3 - protein, 383 aa, Artificial Sequence
  Description of Artificial Sequence Recombinant protein
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIKGGGGSGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 4 - protein, 405 aa, Artificial Sequence
  Description of Artificial Sequence Recombinant protein
MKYLLPTAAAGLLLLAAQPAMAQVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIKGGGGSGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 5 - DNA, 1218 bp, Artificial Sequence
  Description of Artificial Sequence Recombinant DNA
atgaaatatctgctgccgaccgcagcagcgggtctgctgctgctggcagcacagcctgcaatggcacaggttaaactggaacagagcggtgccgaagttgttaaaccgggtgcaagcgttaaactgagctgtaaagcaagcggctttaacatcaaagatagctatatgcattggctgcgtcagggtccgggtcagcgtctggaatggattggttggattgatccggaaaatggtgataccgaatatgcaccgaaatttcagggtaaagcaacctttaccaccgataccagcgcaaataccgcatatctgggtctgagcagcctgcgtccggaagataccgcagtgtattattgtaatgaaggcaccccgaccggtccgtattatttcgattattggggtcagggcaccctggttaccgttagcagcggtggtggtggtagtggtggcggtggttcaggcggtggcggtagcgaaaatgttctgacccagagcccgagcagcatgagcgttagcgttggtgatcgtgttaatattgcatgtagcgcaagcagcagcgttccgtacatgcactggctgcagcagaaaccgggtaaaagcccgaaactgctgatttatctgaccagcaatctggcaagcggtgttccgagccgttttagcggtagcggtagtggcaccgattatagcctgaccattagcagcgtgcagcctgaagatgcagcaacctattattgtcagcagcgtagcagttatccgctgacctttggtggtggcaccaaactggaaattaaagggggtggtggctcaggtggcggaggtgcagaagcaggtattaccggtacatggtcagatcagctgggtgatacctttattgttaccgcaggcgcagatggtgcactgaccggcacctatgaaaatgcagttggtggtgcagaaagccgttatgtgctgaccggtcgttatgatagcgcaccggcaaccgatggtagcggcaccgcactgggttggaccgttgcatggaaaaataacagcaaaaatgcacatagcgcaaccacctggtcaggtcagtatgtgggtggtgccgatgccaaaattaacacccagtggctgctgaccagcggtacaaccaatgcaaatgcctggaaaagtaccctggttggtcatgatacattcaccaaagttaaaccgagcgcagcaagccatcatcatcaccatcattaa

SEQ ID NO: 6 - protein, 141 aa, Artificial Sequence
  Description of Artificial Sequence Recombinant protein
MDKIAIVNMGSLFQQVAQKTGVSNTLENEFKGRASELQRMETDLQAKMKKLQSMKAGSDRTKLEKDVMAQRQTFAQKAQAFEQDRARRSNEERGKLVTRIQTAVKSVANSQDIDLVVDANAVAYNSSDVKDITADVLKQVK

SEQ ID NO: 7 - protein, 247 aa, Artificial Sequence
  Description of Artificial Sequence Recombinant protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 8 - protein, 391 aa, Artificial Sequence
  Description of Artificial Sequence Recombinant protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGGSGGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 9 - protein, 413 aa, Artificial Sequence
  Description of Artificial Sequence Recombinant protein
MKYLLPTAAAGLLLLAAQPAMAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGGSGGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 10 - DNA, 1242 bp, Artificial Sequence
  Description of Artificial Sequence Recombinant DNA
atgaaatatctgctgccgaccgcagcagcgggtctgctgctgctggcagcacagcctgcaatggcagaagttcagctggttgaaagcggtggtggtctggttcagcctggtggtagcctgcgtctgagctgtgcagcaagcggttttaacattaaagatacctatattcattgggtgcgtcaggcacctggtaaaggtctggaatgggttgcacgtatttatccgaccaatggttatacccgttatgccgatagcgttaaaggtcgttttaccattagcgcagataccagcaaaaataccgcatacctgcagatgaatagtctgcgtgcagaggataccgcagtgtattattgtagccgttggggtggtgatggtttttatgcaatggattattggggtcagggcaccctggttaccgttagctcaggtggaggcggttccggtggcggaggttccggtggaggtggctccggtggcggaggttccgatattcagatgacccagagtccgagcagcctgagcgcaagcgttggtgatcgtgtgaccattacctgtcgtgcaagccaggatgttaatacagcagttgcatggtatcagcagaaaccgggtaaagcaccgaaactgctgatttatagcgcaagctttctgtatagcggtgttccgagccgttttagcggtagccgtagcggcaccgattttaccctgaccattagcagcctgcagccggaagattttgcaacctattattgtcagcagcattacaccacaccgcctacctttggccagggcaccaaagttgaaattaaaggaggtggcggtggatccggcggaggtggcggagcagaagcaggtattaccggtacatggtcagatcagctgggtgatacctttattgttaccgcaggcgcagatggtgcactgaccggcacctatgaaaatgcagttggtggtgcagaaagccgttatgtgctgaccggtcgttatgatagcgcaccggcaaccgatggtagcggcaccgcactgggttggaccgttgcatggaaaaataacagcaaaaatgcacatagcgcaaccacctggtcaggtcagtatgtgggtggtgccgatgccaaaattaacacccagtggctgctgaccagcggtacaaccaatgcaaatgcctggaaaagtaccctggttggtcatgatacattcaccaaagttaaaccgagcgcagcaagccatcatcatcaccatcattaa

SEQ ID NO: 11 - protein, 9 aa, Artificial Sequence
  Description of Artificial Sequence Recombinant linker
GGGGSGGGG

SEQ ID NO: 12 - DNA, 40 bp, Artificial Sequence
  Description of Artificial Sequence Synthesized DNA
aaggagatatacatatggataaaattgccattgttaatat

SEQ ID NO: 13 - DNA, 39 bp, Artificial Sequence
  Description of Artificial Sequence Synthesized DNA
ttgagatctgccatatgttatttcacttgtttcagaacg

SEQ ID NO: 14 - DNA, 35 bp, Artificial Sequence
  Description of Artificial Sequence Synthesized DNA
agaaggagatataccatgaaatatctgctgccgac

SEQ ID NO: 15 - DNA, 36 bp, Artificial Sequence
  Description of Artificial Sequence Synthesized DNA
cgccgagctcgaattttaatgatggtgatgatgatg

SEQ ID NO: 16 - DNA, 26 bp, Artificial Sequence
  Description of Artificial Sequence Synthesized DNA
ggtatatctccttcttaaagttaaac

SEQ ID NO: 17 - DNA, 24 bp, Artificial Sequence
  Description of Artificial Sequence Synthesized DNA
aattcgagctcggcgcgcctgcag

SEQ ID NO: 18 - protein, 11 aa, Artificial Sequence
  Description of Artificial Sequence Recombinant linker
GGGGGSGGGGG

SEQ ID NO: 19 - protein, 127 aa, Artificial Sequence
  Description of Artificial Sequence Recombinant peptide
AEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAAS